(19) United States
(12) Patent Application Publication
(10) Pub. No.: US 2005/0153913 A1
(43) Pub. Date: Jul. 14, 2005

(54) Nucleic acid carrier compositions and methods for their synthesis

(76) Inventor: Kenneth M. Kosak, West Valley City, UT (US)

Correspondence Address: KENNETH M. KOSAK, 3194 S. 4400 W., West Valley City, UT, 84120, US

(21) Appl. No.: 10/878,175

(22) Filed: Jun. 28, 2004

Related U.S. Application Data
(63) Continuation-in-part of application No. 09/829,551, filed on Apr. 10, 2001, now Pat. No. 6,835,718.

(51) Int. Cl. A61K 48/00

(52) U.S. Cl. 514/44

(57) ABSTRACT

This invention discloses compositions and methods for preparing pharmaceutical nucleic acid carriers. The compositions comprise a carrier substance coupled to a nucleic acid intercalator whereby the intercalator is coupled by intercalation to the nucleic acid. The compositions can also include a biocleavable linkage for carrying and releasing nucleic acids for therapeutic or other medical uses. The invention also discloses nucleic acid carrier compositions that are coupled to targeting molecules for targeting the delivery of nucleic acids to their site of action.

### RELATED PATENT APPLICATION

This is a continuation-in-part application of U.S. patent application Ser. No. 09/829,551, filed Apr. 10, 2001. The contents of that application are incorporated herein.

### TECHNICAL FIELD OF THE INVENTION

This invention discloses pharmaceutical nucleic acid carrier compositions that include covalent and noncovalent linkages between nucleic acids and various carrier substances. The carrier substances include polysaccharides, synthetic polymers, proteins, micelles and other substances for carrying and releasing the nucleic acids into the body for therapeutic effect.

Specifically, the invention is a nucleic acid carrier composition comprised of a carrier substance coupled to a nucleic acid intercalator. The nucleic acids, such as antisense oligodeoxynucleotides, are thereby coupled through intercalation to the intercalator and carrier substance. The carrier compositions can contain biocleavable linkages that release the nucleic acids under controlled conditions. The carrier compositions can also be coupled to targeting molecules for targeting the delivery of nucleic acids to their site of action. The invention also discloses methods for preparing nucleic acid carrier compositions.

### DESCRIPTION OF THE PRIOR ART

Nucleic acid therapies such as gene therapy and especially antisense nucleic acid therapy hold great promise for the treatment of many diseases and gene-related disorders. However, when nucleic acids are administered in their “free” form, they suffer from low uptake rate by target cells. Also, free nucleic acids are subjected to dilution, nonspecific binding and degradation in the bloodstream. Because of these reasons, carriers for nucleic acids have gained acceptance as a way of solving these problems and improving nucleic acid therapies. However, there is a need for more simplified coupling methods between nucleic acids and the carrier substances and/or targeting moieties that facilitate the delivery of nucleic acids into the body and improve their effectiveness.

In the prior art, nucleic acids have been encapsulated into liposomes, where nucleic acids are protected from serum nuclease degradation. Nucleic acids have also been attached to cationic substance such as polyethylenimine (PEI) or hydrophobic moieties, such as cationic lipids, cholesterol, or geraniol. These coupling methods rely upon hydrophobic interactions and/or “salt bonds” where negatively charged nucleic acids are attracted to positively charged carrier.

Wang, et al, Patent Applic. # U.S. 2003/0144222 A1, discloses an interesting approach using cyclodextrin monomers couple to polyethylene glycol (PEG). The resulting pendant cyclodextrin monomers are then employed as noncovalent complexing agents to entrap oligonucleotides and other drugs. Their method suffers from the same problems as other conventional noncovalent systems in that there is low stability.

The prior art now employs a variety of chemistries for covalent coupling of nucleic acids to carriers that include synthetic polymers such as PEG. Such carriers may also include targeting moieties such as antibodies, polypeptides and other substances to re-direct antisense oligonucleotides and other nucleic acids to selected target cells.

Because nucleic acids generally do not contain convenient coupling sites, the prior art requires that they be incorporated into the nucleic acid during synthesis or added through derivatization after synthesis. Similarly, the carrier substance must also contain a suitable coupling site, which generally must be added through derivatization. Then, in order to couple the derivatized nucleic acid to the carrier substance, a suitable cross linking agent is needed to covalently couple with the sites on the nucleic acid and carrier substance. When the coupling sites are similar, this approach allows a certain percentage of self-coupling of the nucleic acids or the carrier substance, which is inefficient and more costly.

Alternatively, the prior art has used carriers with specific reactive groups for coupling to nucleic acids. This also requires derivatizing the nucleic acid and limits the choices to functional groups that are compatible with the carrier reactive group. Another problem is that before or after coupling the nucleic acid to the carrier, other moieties such as a targeting moiety, may need to be coupled. This frequently causes more limitations since the method used for coupling other moieties must be specific for that moiety and not interfere with or compete with the nucleic acid coupling method. This frequently requires more complicated and costly synthesis methods. For instance, protecting and deprotecting groups are usually needed to avoid adverse reactions such as inactivation of the nucleic acid.

It will be apparent that the compositions of the instant invention overcome these limitations. The carrier compositions of the present invention contain nucleic acid intercalators that are generally unreactive with most functional groups but will bind specifically with nucleic acids. This invention removes many limitations making it easier to synthesize nucleic acid carriers.

Prior Art Nucleic Acid Intercalators as Labels, Probes and Mutagens.

The most well known nucleic acid intercalator is psoralen, a drug used for generations to treat certain skin diseases including psoriasis and vitiligo. The treatment is usually called PUVA, where the drug is given orally and followed up with short exposure to ultraviolet light.

It is also well known in the art of nucleic acids that certain nucleic acid intercalators have many uses that are generally limited to in vitro labeling, nucleic acid probes and mutagenic applications. For instance, several suppliers provide psoralen reagents for incorporating amino groups or fluorescent labels into nucleic acid. The psoralen is covalently coupled to the nucleic acid by photoactivation. However, there are no apparent references for coupling psoralen to the carrier substances of the present invention or for using psoralen in the disclosed compositions.

The prior art also discloses intercalators including psoralen for use in probing specific sequences or binding sites of proteins and nucleic acids. These include compositions where psoralen has been incorporated into nucleic acid sequences of various probes for sequence recognition and binding. For instance, M. Kurz, in U.S. patent application No. 20030100004, discloses the use of psoralen in cross linking methods for preparing immobilized peptide or protein on a solid support. The present invention is directed to the art of nucleic acid delivery and eliminates any need for sequence recognition by the carrier.

Also, the prior art discloses mutagenic nucleic acids containing intercalators that, when hybridized to their target, the mutagen is proximal to the site in the targeted DNA or RNA requiring modification. For instance P. M. Glazer, et al, U.S. patent application No. 20020028922, discloses mutagenic triplex-forming oligonucleotides using an intercalator such as psoralen to alter the function of hybridized target molecules. Such prior art compositions require that the intercalator be coupled to a nucleic acid or peptide that must first recognize a certain sequence, then hybridize with the target molecule in order to function as a useful mutagen. The carrier compositions of the present invention do not provide the critical hybridization step and therefore, are useless in the mutagenic nucleic acids of the prior art.

The prior art discloses many synthesis methods for incorporating psoralen into nucleic acids. For example, commercially available compounds such as psoralen C2 phosphoramidite (Glen Research, Sterling, Va.) are inserted into a specific location within an oligonucleotide sequence in accordance with the methods of Takasugi et al., Proc. Natl. Acad. Sci. U.S.A. 88: 5602-5606 (1991), Gia et al., Biochemistry 31: 11818-11822 (1992), Giovannangeli et al., Nucleic Acids Res. 20: 4275-4281 (1992) and Giovannangeli et al., Proc. Natl. Acad. Sci. U.S.A. 89: 8631-8635 (1992), all of which are incorporated by reference herein.

The intercalator may also be attached to the nucleic acid by a covalent linker, such as sulfo-m-maleimidobenzoly-N-hydroxysuccinimide ester (sulfo-MBS, Pierce Chemical Co., Rockford, Ill.) in accordance with the methods of Liu et al., Biochem. 18: 690-697 (1979) and Kitagawa and Ailawa, J. Biochem. 79: 233-236 (1976), both of which are incorporated by reference herein.

The prior art disclosures of psoralen and other intercalators are as research tools for actively labeling, probing and synthesizing micro arrays. They are directed toward solving different problems in their respective fields unrelated to the pharmaceutical compositions of this invention. The prior art compositions require that the intercalator be still “active” or available for intercalation when used as a probe or mutagen.

In the compositions of this invention, the intercalators, such as psoralen, are intercalated into the composition before use. The present compositions cannot be used in the prior art intercalator compositions since the intercalator has already been consumed during synthesis of the loaded carrier. Conversely, the prior art compositions with sequence recognition and active intercalators are missing the crucial nucleic acid for delivery.

Apparently, the only pharmaceutical applications for psoralen in the prior art are for PUVA treatment and in mutagenic nucleic acid applications. Surprisingly, as well known as psoralen is, there is no disclosure or suggestion for using psoralens or other intercalators as is disclosed in the pharmaceutical carrier compositions of the present invention. Also, it has been discovered that there are unexpected advantages in the coupling compositions of the instant invention.

### SUMMARY OF THE INVENTION

It will be understood in the art of nucleic acids that there are limitations as to which derivatives, coupling agents or other substances can be used with nucleic acids to fulfill their intended function. The terms “suitable” and “appropriate” refer to substances or synthesis methods known to those skilled in the art that are needed to perform the described reaction or to fulfill the intended function. It will also be understood in the art of nucleic acids and drug carriers that there are many substances defined herein that, under specific conditions, can fulfill more than one function. Therefore, if they are listed or defined in more than one category, it is understood that each definition depends upon the conditions of their intended use.

The present invention is a nucleic acid carrier composition comprised of a carrier substance covalently coupled to a nucleic acid intercalator. Before use, the carrier composition is loaded with the desired nucleic acid by combining the carrier and the nucleic acid under relatively mild conditions that allow intercalation with the nucleic acid.

The carrier substance can include a variety of suitable substances including proteins, carbohydrates, polymers, grafted polymers and amphiphilic molecules disclosed herein. The nucleic acid intercalator can include a biodegradable linkage between the intercalator and the carrier substance to provide controlled release of the intercalated nucleic acid after the carrier has reached its site of action. Optionally, one or several moieties can also be coupled to the carrier such as targeting molecules for targeting and transduction vectors disclosed herein to provide other desirable properties.

Any suitable synthesis method now used for preparing polymers conjugated to various moieties, with suitable modification, is applicable to the synthesis of this invention. A distinguishing property of this invention is that the nucleic acid coupling component is able to intercalate with a nucleic acid.

Suitable polymers such as polyethylene glycol are commercially available in a variety of molecular masses. Based on their molecular size, they are arbitrarily classified into low molecular weight (Mw<20,000) and high molecular weight (Mw>20,000). In this invention, polymers of a molecular weight of 20,000 or greater are preferred when the purpose is to prevent rapid elimination of the polymer-coupled nucleic acid due to renal clearance.

In one preferred embodiment, a suitable polyethylene glycol carrier has pendant reactive groups. The reactive groups are suitably conjugated to one or more nucleic acid intercalating moieties using various bifunctional cross-linking agents. The preferred embodiment may include biocleavable linkages as described herein. In another preferred embodiment, the carrier is suitably targeted by coupling suitable biorecognition molecules to the polymer carrier.

It has been discovered that the nucleic acid coupling compositions in the instant invention overcome many limitations of other coupling systems in the prior art. The instant invention thereby provides new properties and unexpected advantages.

### DESCRIPTION OF THE PREFERRED EMBODIMENTS

For the purposes of disclosing this invention, certain words, phrases and terms used herein are defined below.

### Active Agents

Small Molecular Active Agents.

Small molecular active agents (or “small active agents” or “small drugs”), are defined here as chemicals and other substances with a molecular weight usually less than 1500 Daltons and are inhibitory, antimetabolic, therapeutic or preventive toward any disease (i.e. cancer, viral diseases, bacterial diseases and heart disease) or inhibitory or toxic toward any disease causing organism. Preferred small active agents are any therapeutic small drugs categorized in The Merck Index, Eleventh Ed., Merck & Co. Inc., Rahway N.J. (1989) and those listed by Cserhati, T., Anal. Biochem. 225(2), 328-332 (1995).

Small active agents include but are not limited to therapeutic small drugs that include prodrugs, anticancer small drugs, antineoplastic small drugs, antifungal small drugs, antibacterial small drugs, antiviral small drugs, cardiac small drugs, neurological small drugs, and small drugs of abuse; alkaloids, antibiotics, steroids, steroid hormones, narcotics, pesticides and prostaglandins.

Small active agents also include any small toxins including aflatoxins, irinotecan, ganciclovir, furosemide, indomethacin, chlorpromazine, methotrexate, cevine derivatives and analogs including cevadines, desatrines, and veratridine, among others.

Small active agents that are also included but not limited to, are;
various antibiotics including derivatives and analogs such as penicillin derivatives (i.e. ampicillin), anthracyclines (i.e. doxorubicin, daunorubicin, mitoxantrone), butoconazole, camptothecin, chalcomycin, chartreusin, chrysomicins (V and M), chloramphenicol, chlorotetracyclines, clomocyclines, ellipticines, filipins, fungichromins, griseofulvin, griseoviridin, guamecyclines, macrolides (i.e. amphotericins, chlorothricin), methicillins, nystatins, chrymutasins, elsamicin, gilvocarin, ravidomycin, lankacidin-group antibiotics (i.e. lankamycin), mitomycin, teramycins, tetracyclines, wortmannins;
various anti-microbials including reserpine, spironolactone, sulfacetamide sodium, sulphonamide, thiamphenicols, thiolutins;
various purine and pyrimidine derivatives and analogs including 5′-fluorouracil, 5′-fluoro-2′-deoxyuridine, and allopurinol;
various steroidal compounds such as cortisones, estradiols, hydrocortisone, dehydroepiandrosterone (DHEA), testosterone, prednisolones, progesterones, dexamethasones, beclomethasones and other methasone derivatives, other steroid derivatives and analogs including digitoxins, digoxins, digoxigenins;
various antineoplastic agents or cell growth inhibitors such as cisplatins and taxanes including paclitaxel and docetaxel;

Other small active agents that are included, but are not limited to, are;
vitamins A, B12, C, D3, E, K3, and folic acid, among others.

Protein and Peptide Active Agents.

Protein and peptide active agents are defined here as various proteins, peptides, bioactive peptides and polypeptides that are inhibitory, antimetabolic, therapeutic or preventive toward any disease (i.e. cancer, syphilis, gonorrhea, influenza and heart disease) or inhibitory or toxic toward any disease causing agent. They include polypeptide hormones, interferons, interleukins, laminin fragments, tumor necrosis factors (TNF), cyclosporins, ricins, tyrocidines and bungarotoxins, among others.

Preferred protein and peptide active agents include pro-apoptotic peptides including the mitochondrial polypeptide called Smac/Diablo, or a region from the pro-apoptotic proteins called the BH3 domain and other pro-apoptotic peptides.

Biocompatible.

Biocompatible is defined here to mean substances that are suitably nonimmunogenic, no allergenic and will cause minimum undesired physiological reaction. They may or may not be degraded biologically and they are suitably “biologically neutral” for pharmaceutical applications due to very low specific binding properties or biorecognition properties.

Coupling.

For the instant invention, two distinct types of coupling are defined. One type of coupling can be through noncovalent, “attractive” binding as with a guest molecule and cyclodextrin, an intercalator and nucleic acid, an antigen and antibody or biotin and avidin. Such noncovalent coupling is binding between substances through ionic or hydrogen bonding or van der waals forces, and/or their hydrophobic or hydrophilic properties.

Unless stated otherwise, the preferred coupling used in the instant invention is through covalent, electron-pair bonds or linkages. Many methods and agents for covalently coupling (or cross linking) of carrier substances including polyethylene glycol and other polymers are known and, with appropriate modification, can be used to couple the desired substances through their “functional groups” for use in this invention. Where stability is desired, the preferred covalent linkages are amide bonds, peptide bonds, ether bonds, and thio ether bonds, among others.

Functional Group.

A functional group or reactive group is defined here as a potentially reactive moiety or “coupling site” on a substance where one or more atoms are available for covalent coupling to some other substance. When needed, functional groups can be added to a carrier substance such as polyethylene glycol through derivatization or substitution reactions.

Examples of functional groups are aldehydes, allyls, amines, amides, azides, carboxyls, carbonyls, epoxys (oxiranes), ethynyls, hydroxyls, phenolic hydroxyls, indoles, ketones, certain metals, nitrenes, phosphates, propargyls, sulfhydryls, sulfonyls, vinyls, bromines, chlorines, iodines, and others. The prior art has shown that most, if not all of these functional groups can be incorporated into or added to the carrier substances of this invention.

Pendant Functional Group.

A pendant or “branched” functional or reactive group is defined here as a functional group or potentially reactive moiety described herein, that is located on a suitable polymer backbone such as pendant polyethylene glycol between the two ends. Preferably the pendant functional groups are located more centrally than peripherally.

Linkage.

A linkage is defined as a chemical moiety within the compositions disclosed that results from covalent coupling or bonding of the substances disclosed to each other. A linkage may be either biodegradable or non-biodegradable and may contain suitable “spacers” defined herein. Suitable linkages are more specifically defined below.

Coupling Agent.

A coupling agent (or cross-linking agent), is defined as a chemical substance that reacts with functional groups on substances to produce a covalent coupling, or linkage, or conjugation with said substances. Because of the stability of covalent coupling, this is the preferred method. Depending on the chemical makeup or functional group on a carrier substance, amphiphilic molecule, cyclodextrin, or targeting molecule, the appropriate coupling agent is used to provide the necessary active functional group or to react with the functional group. In certain preparations of the instant invention, coupling agents are needed that also provide a linkage with a “spacer” or “spacer arm” as described by O'Carra, P., et al, FEBS Lett. 43, 169 (1974) between a carrier substance and an intercalator or targeting molecule to overcome steric hindrance. Preferably, the spacer is a substance of 4 or more carbon atoms in length and can include aliphatic, aromatic and heterocyclic structures.

With appropriate modifications by one skilled in the art, the coupling methods referenced in U.S. Pat. No. 6,048,736 and PCT/US99/30820, including references contained therein, are applicable to the synthesis of the preparations and components of the instant invention and are hereby incorporated by reference.

Examples of energy activated coupling agents are ultraviolet (UV), visible and radioactive radiation that can promote coupling or cross linking of suitably derivatized substances. Examples are photochemical coupling agents disclosed in U.S. Pat. No. 4,737,454, among others. Also useful in synthesizing components of the instant invention are enzymes that produce covalent coupling such as nucleic acid polymerases and ligases, among others.

Useful derivatizing and/or coupling agents for preparing polymers are bifunctional, trifunctional or polyfunctional cross linking agents that will covalently couple to the functional groups of suitable monomers and other substances.

Useful in this invention are coupling agents selected from the group of oxiranes and epoxides. Some preferred examples of oxiranes and epoxides include; epichlorohydrin, 1,4 butanediol diglycidyl ether (BDDE), bis(2,3-epoxycyclopentyl) ether 2,2′-oxybis(6-oxabicyclo[3.1.0]hexane) (13ECPE), glycerol diglycidyl ether (GDE), trimethylolpropane triglycidyl ether (TMTE), tris(2,3-epoxypropyl) isocyanurate (rEPIC), glycerol propoxylate triglycidyl ether (GPTE), 1,3-butadiene diepoxide, triphenylolmethane triglycidyl ether, 4,4′-methylenebis (N,N-diglycidylaniline), tetraphenylolethane glycidyl ether, bisphenol A diglycidyl ether, bisphenol A propoxylate diglycidyl ether, bisphenol F diglycidyl ether, cyclohexanedimethanol diglycidyl ether, 2,2′-oxybis (6-oxabicyclo[3.1.0] hexane), polyoxyethylene bis(glycidyl ether), resorcinol diglycidyl ether, ethylene glycol diglycidyl ether (EGDE) and low molecular weight forms of poly(ethylene glycol) diglycidyl ethers or poly(propylene glycol) diglycidyl ethers, among others.

Other preferred derivatizing and/or coupling agents for hydroxyl groups are various disulfonyl compounds such as benzene-1,3-disulfonyl chloride and 4,4′-biphenyl disulfonyl chloride and also divinyl sulfone (J. Porath, et al, J. Chromatog. 103, 49-62, 1975), among others.

Most preferred coupling agents are also chemical substances that can provide the bio-compatible linkages for synthesizing the nucleic acid carriers of the instant invention. Covalent coupling or conjugation can be done through functional groups using coupling agents such as glutaraldehyde, formaldehyde, cyanogen bromide, azides, p-benzoquinone, maleic or succinic anhydrides, carbodiimides, ethyl chloroformate, dipyridyl disulfide and polyaldehydes.

Also most preferred are derivatizing and/or coupling agents that couple to thiol groups (“thiol-reactive”) such as agents with any maleimide, vinylsulfonyl, bromoacetal or iodoacetal groups, including any bifunctional or polyfunctional forms. Examples are m-maleimidobenzoyl-N-hydroxysuccinmide ester (MBS), succinimidyl 4-(N-maleimidomethyl) cyclohexane-1-carboxylate (SMCC), succinimidyl 4(p-maleimidophenyl)butyrate (SMPB), dithiobis-N-ethylmaleimide (DTEM), 1,1′-(methylenedi-4,1-phenylene) bismaleimide (MPBM), o-phenylenebismaleimide, N-succinimidyl iodoacetate (SIA), N-succinimidyl-(4-vinylsulfonyl) benzoate (SVSB), and tris-(2-maleimidoethyl) amine (TMEA), among others.

Other coupling groups or agents useful in the instant invention are: p-nitrophenyl ester (ONp), bifunctional imidoesters such as dimethyl adipimtidate (DMA), dimethyl pimelimidate (DMP), dimethyl suberimidate (DMS), methyl 4-mercaptobutyrimidate, dimethyl 3,3′-dithiobispropionimidate (DTBP), and 2-iminothiolane (Traut's reagent);
bifunctional tetrafluorophenyl esters (TFP) and bifunctional NHS esters such as disuccinimidyl suberate (DSS), bis[2-(succinimido-oxycarbonyloxy) ethyl]sulfone (BSOCOES), disuccinimidyl (N,N′-diacetylhomocystein) (DSAH), disuccinimidyl tartrate (DST), dithiobis(succinimidyl propionate) (DSP), and ethylene glycol bis(succinimidyl succinate) (EGS), including various derivatives such as their sulfo-forms;
heterobifunctional reagents such as p-nitrophenyl 2-diazo-3,3,3-trifluoropropionate, N-succinimidyl-6(4′-azido-2′-nitrophenylamino) hexanoate (Lomant's reagent II), and N-succinimidyl-3-(2-pyridyldithio)propionate (SPDP), including various derivatives such as their sulfo-forms;
homobifunctional reagents such as 1,5-difluoro-2,4-dinitrobenzene, 4,4′-difluoro-3,3′-dinitrophenylsulfone, 4,4′-diisothiocyano-2,2′-disulfonic acid stilbene (DIDS), p-phenylene-diisothiocyanate (DITC), carbonylbis(L-methionine p-nitrophenyl ester), 4,4′-dithio-bisphenylazide and erythritolbiscarbonate, including derivatives such as their sulfo-forms;
photoactive coupling agents such as N-5-azido-2-nitrobenzoylsuccinimide (ANB-NOS), p-azidophenacyl bromide (APB), p-azidophenyl glyoxal (APG), N-(4-azidophenylthio) phthalimide (APTP), 4,4′-dithio-bis-phenylazide (DTBPA), ethyl 4-azidophenyl-1,4-dithiobutyrimidate (EADB), 4-fluoro-3-nitrophenyl azide (FNPA), N-hydroxysuccinimidyl-4-azidobenzoate (HSAB), N-hydroxysuccinimidyl-4-azidosalicylic acid (NHS-ASA), methyl-4-azidobenzoimidate (MABI), p-nitrophenyl-2-diazo-3,3,3-trifluoropropionate (PNP-DTP), 2-diazo-3,3,3-trifluoropropionyl chloride, N-succinimidyl-6(4′-azido-2′-nitrophenylamino) hexanoate (SANPAH), N-succinimidyl(4-azidophenyl)1,3′-dithiopropionate (SADP), sulfosuccinimidyl-2-(m-azido-o-nitobenzamido)-ethyl-1,3′-dithiopropionate (SAND), sulfosuccinimidyl (4-azidophenyldithio) propionate (Sulfo-SADP), sulfosuccinimidyl-6-(4′-azido-2′-nitrophenylamino) hexanoate (Sulfo-SANPAH), sulfosuccinimdyl-2-(p-azidosalicylamido) ethyl-1,3′-dithiopropionate (SASD), and derivatives and analogs of these reagents, among others. The structures and references for use are given for many of these reagents in, “Pierce Handbook and General Catalog”, Pierce Chemical Co., Rockford, Ill., 61105.

Biocleavable Linkage or Bond.

For the instant invention, biocleavable linkages are defined as types of specific chemical moieties or groups that can be used within the chemical substances that covalently couple or cross-link a carrier substance with the intercalators, active agents, targeting moieties, amphiphilic molecules and grafted polymers described herein. They may also be contained in certain embodiments of the instant invention that provide the function of controlled release of nucleic acids. Biocleavable linkages or bonds are distinguishable by their structure and function and are defined here under distinct categories or types.

One category comprises the disulfide linkages that are well known for covalent coupling. For drug delivery, they may be more useful for shorter periods in vivo since they are cleaved in the bloodstream relatively easily. The simple ester bond is another preferred type that includes those between any acid and alcohol. Another preferred type is any imidoester formed from alkyl imidates. Also included are maleimide bonds as with sulfhydryls or amines used to incorporate a biocleavable linkage.

Another category in this invention comprises biocleavable linkages that are more specifically cleaved after entering the cell (intracellular cleavage). The preferred biocleavable linkages for release of active agents and other moieties within the cell are cleavable in acidic conditions like those found in lysosomes. One type is an acid-sensitive (or acid-labile) hydrazone linkage as described by Greenfield, et al, Cancer Res. 50, 6600-6607 (1990), and references therein.

Another type of preferred acid-labile linkage is any type of polyortho or diortho ester linkage, examples disclosed by J. Heller, et al., Methods in Enzymology 112, 422-436 (1985), J. Heller, J. Adv. Polymer Sci. 107, 41 (1993), M. Ahmad, et al., J. Amer. Chem. Soc. 101, 2669 (1979) and references therein. Also preferred are acid labile phosphonamide linkages disclosed by J. Rahil, et al, J. Am. Chem. Soc. 103, 1723 (1981) and J. H. Jeong, et al, Bioconj. Chem. 14, 473 (2003).

Another preferred category of biocleavable linkages is biocleavable peptides or polypeptides from 2 to 100 residues in length, preferably from 3 to 20 residues in length. These are defined as certain natural or synthetic polypeptides that contain certain amino acid sequences (i.e. are usually hydrophobic) that are cleaved by specific enzymes such as cathepsins, found primarily inside the cell (intracellular enzymes). Using the convention of starting with the amino or “N” terminus on the left and the carboxyl or “C” terminus on the right, some examples are: any peptides that contain the sequence Phe-Leu, Leu-Phe or Phe-Phe, such as Gly-Phe-Leu-Gly (GFLG), Gly-Phe-Leu-Phe-Gly and Gly-Phe-Phe-Gly, and others that have either of the Gly residues substituted for one or more other peptides. Also included are leucine enkephalin derivatives such as Tyr-Gly-Gly-Phe-Leu.

Biocleavable peptides also include cathepsin cleavable peptides such as those disclosed by J. J. Peterson, et al, in Bioconj. Chem., Vol. 10, 553-557, (1999), and references therein. Some examples are; GGGF, GFQGVQFAGF, GFGSVQFAGF, GFGSTFFAGF, GLVGGAGAGF, GGFLGLGAGF and most preferred are GFQGVQFAGF, GFGSVQFAGF, GLVGGAGAGF, GGFLGLGAGF, and GFGSTFFAGF. Also preferred are any peptides that contain parts of these cathepsin cleavable sequences.

Another preferred type of biocleavable linkage is any disulfide linkages such as those produced by thiol-disulfide interchange (J. Carlsson, et al, Eur. J. Biochem. 59, 567-572, 1975). Another preferred type of biocleavable linkage is any “hindered” or “protected” disulfide bond that sterically inhibits attack from thiolate ions. Examples of such protected disulfide bonds are found in the coupling agents: S-4-succinimidyl-oxycarbonyl-α-methyl benzyl thiosulfate (SMBT) and 4-succinimidyloxycarbonyl-α-methyl-α-(2-pyridyldithio) toluene (SMPT). Another useful coupling agent resistant to reduction is SPDB disclosed by Worrell, et al., Anticancer Drug Design 1: 179-188 (1986). Also included are certain aryldithio thioimidates, substituted with a methyl or phenyl group adjacent to the disulfide, which include ethyl S-acetyl 3-mercaptobutyrothioimidate (M-AMPT) and 3-(4-carboxyamidophenyldithio) proprionthioimidate (CDPT), disclosed by S. Arpicco, et al., Bioconj. Chem. 8 (3): 327-337 (1997). Another preferred category is certain aldehyde bonds subject to hydrolysis that include various aldehyde-amino bonds (Schiff's base), or aldehyde-sulfhydryl bonds.

Another preferred type of biocleavable linkage in this invention are any suitable aromatic azo linkages that are cleavable by specific azo reductase activities in the colon as disclosed by J. Kopecek, et al., In: Oral Colon Specific Drug Delivery; D. R. Friend, Ed., pp 189-211 (1992), CRC Press, Boca Raton, Fla.

Controlled Release.

For this invention, controlled release (or “active release”) is defined as the release of a nucleic acid or other active agent from the nucleic acid carrier. Release of the active agent is by cleavage of certain biocleavable covalent linkages described herein that were used to couple the active agent to the carrier substance or to synthesize the carrier.

### Carrier Substances

The present invention is a nucleic acid carrier composition comprised of a carrier substance covalently coupled to a nucleic acid intercalator. Preferably the carrier substance provides a biocompatible framework or “backbone” to which are coupled various moieties. The carrier substance can include a variety of suitable substances including proteins, carbohydrates, polymers, grafted polymers and amphiphilic molecules disclosed herein. The carrier substance can also include combinations of these suitable substances.

Protein Carrier Substances.

Preferred protein carrier substances include serum or plasma proteins including albumins, fibrinogens, globulins (thyroglobulins), haptoglobins, histones, protamines and intrinsic factor including their derivatives such as their pegylated forms.

Preferred protein carrier substances include antibodies, including all classes of antibodies, monoclonal antibodies, chimeric antibodies, oxidized antibodies, pegylated antibodies, Fab fractions, fragments and derivatives thereof. Also included are antibodies used for specific cell or tissue targeting including antibodies that bind to cell receptors such as anti-transferrin antibodies used to cross the blood brain barrier.

Preferred protein carrier substances include naturally occurring receptors, peptide hormones, enzymes, (especially cell surface enzymes such as neuramimidases) and their derivatives such as their pegylated forms. Preferred protein carrier substances include avidins, streptavidins, staphylococcal protein A, protein G and their derivatives such as their pegylated forms.

Peptide Carrier Substances.

Preferred peptide carrier substances include any suitable peptide including the transduction vectors and receptor binding peptides defined herein. For example, the intercalators of this invention can be coupled to the amphipathic peptide KALA as disclosed by T. B. Wyman, et al, in Biochem. 36, 3008-3017 (1997), which may include derivatives and additional moieties as disclosed herein.

Carbohydrate Carrier Substances.

Preferred carbohydrate carrier substances include alginates, amyloses, dextrans, dextran sulfates, chitosans, chitosan derivatives, cyclodextrins, cyclodextrin dimers, trimers and polymers including linear cyclodextrin polymers, gums (i.e. guar or gellan), hyaluronic acids, lectins, hemagglutinins, pectins, trisaccharides including raffinose and pegylated carbohydrates.

Polymer Carrier Substances.

Preferred polymer carrier substances include hydrogels, pendant polyethylene glycol and other grafted polymers defined herein.

Amphiphilic Carrier Substances.

Preferred amphiphilic carrier substances include cholesterol derivatives, gangliosides, lipoproteins including low density lipoproteins (LDL), phospholipids, pegylated phospholipids, and the amphiphilic molecules defined herein.

Liposome Carrier Substances.

Preferred carrier substances include liposomes as defined herein and pegylated liposomes that contain the amphiphilic molecules as well as the protein, carbohydrate and polymer carrier substances defined herein. Said liposomes have the desired intercalators, targeting molecules, grafted polymers and other moieties coupled to the liposome through suitable covalent coupling that includes biocleavable linkages defined herein.

Coupling to the liposome can also be done through coupling a moiety to a suitable anchor substance such as an amphiphilic molecule or derivative, and then insertion of the anchor substance into the membrane, during or after liposome synthesis.

Liposomes are prepared from suitable amphiphilic molecules and the carrier substances of this invention using well known methods. For instance, a suitable method is disclosed by J. J. Wheeler, et al, in Gene Therapy 6, 271-281 (1999). The method employs detergent dialysis wherein the nucleic acid-lipid conjugate of the present invention can be incorporated into any suitable mixture of amphiphilic molecules and suitable detergent. The detergent is then removed by dialysis to produce lipid vesicles containing nucleic acid. This reference and references therein are hereby incorporated into this invention.

Micelle and Nanoparticle Carrier Substances.

Preferred carrier substances include the micelles, nanoparticles and dendrimers defined herein, including their pegylated forms and those that contain the amphiphilic molecules defined herein, as well as the protein, carbohydrate and polymer carrier substances defined herein. Also included are micelles containing PEG, or poly(ethylene oxide) (PEO), or poly(propylene oxide) (PPO) such as those disclosed by S—F. Chang, et al, in Human Gene Therapy 15, 481-493 (2004), and references therein.

Micelles are prepared from block copolymers using well known methods. For instance, a suitable method is disclosed by P. L. Soo, et al, in Langmuir 18, 9996-10004 (2002) for polycaprolactone-block-poly(ethylene oxide). In that method, a suitable mixture of nucleic acid-loaded lipid and the desired block copolymer are prepared in a suitable solvent such as DMF. Micellation is achieved by slowly adding water (2.5%/minute), with constant stirring, until the desired water content is achieved (i.e. 80-99%). The product is purified by exhaustive dialysis against water. This reference and references therein are hereby incorporated into this invention.

### Nucleic Acid Intercalators

A nucleic acid intercalator is defined as a substance that is capable of binding to nucleic acid defined herein, through attractive forces of intercalation including through van der Waals forces and/or hydrophobic attraction. For the purposes of this invention, preferred intercalators are aromatic compounds that bind to single stranded nucleic acid (“hemi-intercalator”) or to double stranded (duplex) nucleic acid or to triple stranded (triplex) nucleic acid.

Nucleic acid intercalators are preferred that have a functional group available that also allows covalent coupling of the intercalator to a carrier substance without adversely affecting the nucleic acid intercalating or nucleic acid binding function of the intercalator. When such a functional group is not present, it can be added through suitable derivatization of the intercalator. There are many types and categories of intercalators as described herein. Therefore, one skilled in the art can appreciate that some categories of intercalators are more preferred for the intended purpose of this invention than others.

### Covalent Coupling Nucleic Acid Intercalators

A covalent coupling nucleic acid intercalator is defined as a substance that, in addition to intercalating with nucleic acid, is also capable of forming covalent bonds with the nucleic acid when activated through a photoreactive or chemical process.

Photoreactive Intercalators.

The most preferred group of covalent coupling intercalators are the photoreactive intercalators including those of the furocoumarin family of compounds as disclosed by G. D. Cimino in Ann. Rev. Biochem. 54, 1151-1193 (1985), incorporated herein by reference.

Some preferred examples of photoreactive intercalators include psoralens, psoralen amines, hydroxyl psoralens (4′-hydroxymethyl psoralens), trioxsalens (4,5′,8-trimethylpsoralens), trioxsalen amines (4′-aminomethyl-4-5′-8-trimethyl psoralens), hydroxyl trioxsalens (4′-hydroxymethyl trioxsalens), methoxsalens, 5-methoxypsoralens, 8-methoxypsoralens, 4′-hydroxymethyl-4,5′,8-trimethylpsoralens, 4′-methoxymethyl-4,5′,8-trimethylpsoralens, 4′-chloromethyl-4,5′,8-trimethylpsoralens and 4′-N-phthalimidomethyl-4,5′,8-trimethylpsoralens.

Also preferred are any suitable amino, vinyl, sulfhydryl or phosphoramidite derivatives of psoralen or trioxsalen including 6-(4′-hydroxymethyl-4,5′,8-trimethylpsoralen) hexyl-1-O-(beta-cyanoethyl-N,N′-diisopropyl) phosphoramidite, among others. A preferred amino derivative is “psoralen amine” available from Sigma-Aldrich, St. Louis, Mo., 2003 Catalog #P 6100.

Also preferred are any suitable derivatives of psoralen or trioxsalen including biotinylated forms as is disclosed by C. Levenson, et al, in Methods in Enzymology 184, 577-583 (1990).

Also preferred are any suitable psoralen or trioxsalen active esters (i.e. N-hydroxysuccinimide, or 4-nitrophenyl), including 4′-[(3-carboxypropionamido)methyl]-4,5′,8-trimethylpsoralen N-hydroxysuccinimide ester, as is disclosed by M. A. Reynolds in Bioconj. Chem. 3, 366-374 (1992).

Also preferred are suitable amino acid derivatives of psoralen or trioxsalen such as aspartic acid-beta-(4′-aminomethyl-4,5′,8-trimethylpsoralen) disclosed by Z. Wang, et al, in JACS 117, 5438 (1995).

Also preferred are any suitable psoralen or trioxsalen derivatized with anhydride, carboxylate, chloroformate, tosylate or isothiocyanate functional groups.

Also preferred are any suitable psoralen or trioxsalen derivatives herein disclosed that include alkyl, or alkyl amino extensions, or spacer groups.

Anthraquinones.

Another category of nucleic acid intercalators includes photoreactive anthraquinone derivatives as disclosed by T. Koch, et al, in Bioconj. Chem. 11, 474-483 (2000).

Nucleic Acid Alkylating Agents.

Another category of nucleic acid intercalators includes alkylating agents such as p-azidophenacyl, duocarmycin A (i.e. pyrinamycins) and duocarmycin C. Also the agent (+)—CC-1065 and its analogs possessing the 1,2,9,9a-tetrahydrocyclopropa [1,2-c]benz [1,2-e]indol-4-one (CBI) alkylation subunit including 1-(chloromethyl)-5-dihydro-3H-benz[e]indole (seco-CBI) disclosed by A. Y. Chang, et al, JACS 122, 4856-4864 (2000), and naphthopyranone epoxides disclosed by K. Nakatani, et al, in JACS 123, 5695-5702 (2001) and references therein.

Another category of nucleic acid intercalators includes certain intercalators known to produce covalent nucleic acid complexes such as aflatoxin B oxide and certain pluramycin antibiotics (i.e. kapuramycin A).

Activated Nucleic Acid-Peptide Cross-Linkers.

Another category of nucleic acid intercalator agents includes activated nucleic acid-peptide cross-linkers, defined as substances that promote cross-lining of protein or peptides with nucleic acid. Generally this is through an energy transfer and/or oxidation step initiated by photoactivation of the intercalator agent in close proximity with the nucleic acid and a peptide. Examples of such intercalator agents are cisplatins and cisplatin analogs, neocarcinostatins, iron(III) bleomycins and certain dipyridophenazine complexes of ruthenium including Ru(1,10-phenanthroline)4-(butyric acid)-4′-methyl-2,2′-bipyridine dipyridophenazine disclosed by K. D. Copeland in Biochem 41, 12785 (2002) and references therein.

### Non-Covalent Coupling Nucleic Acid Intercalators

A non-covalent coupling nucleic acid intercalator is defined as a substance that generally does not form covalent bonds with the nucleic acid, but is coupled through the forces of intercalation. The most preferred non-covalent coupling nucleic acid intercalators are those that form and maintain the strongest noncovalent bonds, especially under physiological or pharmaceutical conditions.

Acridine and Acridine Derivatives.

One category of nucleic acid intercalators includes acridine and acridine derivatives such as acridine orange and derivatives thereof, acridine carboxamides, 9-aniloacridine, 3-(9-acridinyl amino)-5-hydroxyethyl aniline (AHMA) derivatives and their alkylcarbamates, acroycines including 1,2-dihydroxy-1,2-dihydro acronycine and 1,2-dihydroxy-1,2-dihydro benzo[b] acronycine diesters, pyrimidol[5,6,1-de]acridines, pyrimidol[4,5,6,-kl]acridines, bis(amine-functionalized) 9-acridone-4-carboxamides, bis(amine-functionalized) acridine-4-carboxamides and pyrazolo [3,4,5-k1] acridine-5-carboxamides.

Also included are bis-acridines disclosed by May, et al, in PNAS, vol. 100, 3416-3421 (2003), and references therein, including bis-(6-chloro-2-methoxy-acridin-9-yl) and bis-(7-chloro-2-methoxy-benzo[b][1,5]-naphthyridin-10-yl) analogs such as (6-chloro-2-methoxy-acridin-9-yl)-(3-{4-[3-(6-chloro-2-methoxyacridin-9-ylamino)-propyl]-piperazin-1-yl}-propyl)-amine, N,N′-bis-(6-chloro-2-methoxy-acridin-9-yl)-1,8-diamino-3,6-dioxaoctane, and (1-{[4-(6-chloro-2-methoxy-acridin-9-ylamino)-butyl]-[3-(6-chloro-2-methoxy-acridin-9-ylamino)-propyl]-carbamoyl}-ethyl)-carbamic acid tert-butyl ester. Also included are quinacrines and covalent dimers of quinacrine.

Anthracyclines and Derivatives.

Another category of nucleic acid intercalators includes anthracyclines such as nogalamycin, daunomycin and adriamycin (doxorubicin), mitoxantrone and ametantrone. Also included are ene-diyne antibiotics such as dynemycin.

Anthracenes and Derivatives.

Another category of nucleic acid intercalators includes anthracenes, phenylanthracenes and their derivatives, including anthraquinolyns, Actinomycins and Derivatives.

Another category of nucleic acid intercalators includes actinomycins including actinomycins C, actinomycins D, 7-amino actinomycin, mitomycin C,

Aminoglycosides and Derivatives.

Another category of nucleic acid intercalators includes aminoglycosides such as neomycin B, kanamycin A, and tobramycin including derivatives such as their conjugates with 9-aminoacridine as are disclosed by Luedtke, et al, in Biochemistry Vol. 42, 11391-11403 (2003) and references therein. Also included are conjugates neo-N-acridine, neo-C-acridine, tobra-N-acridine, kana-N-acridine, neo-N-neo, tobra-N-tobra, neo-5-acridine, neo-neo, tobra-tobra, and kanaA-kanaA.

Porphyrins.

Another category of nucleic acid intercalators includes porphyrins, hematoporphyrins and derivatives, metal-free porphyrins such as H2TMpyP-4. Also included are four-coordinate metalloporphyrins such as CuTMpyP-4, NiTMpyP-4 and PdTMpyP-4 and [Ru(II)12S4dppz]Cl₂.

Pyrenes and Other Intercalators.

Another category of nucleic acid intercalators includes suitable pyrene intercalators including 1-O-(1-pyrenylmethyl)glycerol and derivatives thereof. Another category of nucleic acid intercalators includes ethidiums, propidiums, proflavins, ellipticines and 4,6′-diaminide-2-phenylindole (DAPI).

Another category of nucleic acid intercalators includes distamycin, berenil, Hoechst dyes including Hoechst 33258 and Hoechst 33342.

Covalent Intercalation Linkage.

A covalent intercalation linkage is defined for this invention as a composition wherein an intercalator is a fully covalent coupling agent between a nucleic acid and a carrier substance defined herein. Said intercalator is covalently coupled to said carrier substance through suitable functional groups and/or through a covalent cross linking agent and also covalently coupled through “covalent intercalation” to said nucleic acid. Said covalent intercalation comprises intercalation with said nucleic acid and subsequent conversion of the intercalation binding to a covalent bond or coupling through chemical or photochemical means.

Non-Covalent Intercalation Linkage.

A noncovalent intercalation linkage is defined for this invention as a composition wherein an intercalator can be covalently coupled as defined to said carrier substance but is noncovalently coupled only through the forces of intercalation to said nucleic acid.

Non Pharmaceutical Nucleic Acids.

It is well known in the art of nucleic acids that certain nucleic acids are only useful for in vitro applications. Such nucleic acids and compositions containing them are designed for in vitro applications and are non-pharmaceutical in that they have no potential use in pharmaceutical applications. Therefore, they are unsuitable for the purposes of this invention. Unsuitable nucleic acids are non-pharmaceutical nucleic acid primers and probes designed and used exclusively in the polymerase chain reaction (PCR), nucleic acid sequencing, hybridization methods including Western blots and various micro array probes. However, any nucleic acids with known or potential pharmaceutical value are preferred and useful in this invention even though they may also be used, evaluated, characterized or screened in various in vitro methods.

### Nucleic Acids

For the purposes of this invention, “nucleic acids” are defined as any nucleic acids useful or potentially useful in a pharmaceutical or therapeutic application in humans or any other vertebrate animal and in plants. The most preferred nucleic acids defined as pharmaceutical are nucleic acid active agents against viral and other microbial diseases, against cancers, heart diseases, autoimmune diseases, genetic and other diseases in humans and other vertebrates. Also included are nucleic acid active agents against viral and other microbial diseases in plants. They also include specific DNA sequences used for gene therapy.

RNA

Nucleic acid active agents can include all types of single stranded or double stranded RNA (dsRNA), including antisense RNA, messenger RNA (mRNA) and transfer RNA (tRNA). Most preferred are any RNAs useful in RNA interference (RNAi) therapeutics such as small interfering RNAs (siRNA) and interfering dsRNA.

In one preferred embodiment for coupling dsRNA, the desired sense RNA single strand is first coupled by intercalation to a suitable carrier, then the antisense strand is hybridized with the sense strand on the carrier to form dsRNA. Alternatively, the desired sense RNA single strand is hybridized with the antisense strand to form dsRNA, which is then coupled by intercalation to a suitable carrier.

Examples of preferred RNA in this invention are the following sequence compositions;

RNA sequence A with Amino is:

5′-UGU GGA UGA CUG AGU ACC UGA dTdT-Amino-3′

RNA sequence B is:

5′-UCA GGU ACU CAG UCA UCC ACA dTdT-3′

Also preferred are any micro RNAs (miRNA) and any antisense nucleic acids used to inactivate mRNA, such as antisense nucleic acids containing 2′-O-methyl groups, including those disclosed by Hutvagner, et al, PLOS Biol. 2, 10. 10371/Journal.pbio.0020114(2004) and Meister, et al, RNA 10, 544 (2004).

Also preferred nucleic acids are any ribozymes and hairpin ribozymes including those disclosed or referenced by Y. Lian, et al, in Gene Therapy, Vol. 6, 1114-1119 (1999).

Also preferred nucleic acids are any suitable plasmids and pCOR plasmids including those disclosed or referenced by F. Soubrier, et al, in Gene Therapy, Vol. 6, 1482-1488 (1999).

Also preferred are any riboswitches. Riboswitches are defined as metabolite-binding nucleic acids, or specific metabolite-binding nucleic acid sequences, such as in messenger RNAs that serve as sensors for modulation of gene expression or other functions. Some examples are described by M. Mandal, et al, Cell 113, 577 (2003), including references therein, all of which are incorporated by reference herein. Preferred riboswitches, or the specific metabolite-binding nucleic acid sequences, are those found in vertebrates, mammalian cells, bacteria and higher plants.

Also preferred are 5′ derivatized RNA, or 3′ derivatized RNA where the 5′ or 3′ ends have been capped, or labeled, or extended with additional nucleic acids, or amino acids, or a mutagen, or suitably derivatized in any way. Also preferred are “backbone derivatized” RNAs in which the sugar-phosphate “backbone” has been derivatized or replaced with “backbone analogues” which include phosphorothioate, phosphorodithioate, phosphoroamidate, alkyl phosphotriester, or methylphosphonate linkages or other backbone analogues. Such derivatized RNA includes any sense or antisense sequences.

The two strands of the siRNA duplex can be produced by standard protocols, and many of the chemical modifications that have been developed to improve classical antisense oligonucleotides can also be introduced into RNA (Braasch, D. A., et al, Biochem. 42, 7967, 2003). These modifications may improve the thermal stability, serum stability, cellular activity, or pharmacokinetic properties of RNA. Nucleic acids also include the proteins that make up the RNAi induced silencing complex (RISC).

Also preferred are any “modified ribose” nucleic acids which includes modification of the 2′ position of the ribose ring, including 2′-O-methyl (i.e. 2′-O-meRNA) (Monia, B. P., et al, (1993) J. Biol. Chem. 268, 14514), 2′-deoxy-2′-fluorouridine (Kawasaki, A. M., et al, (1993) J. Med. Chem. 36, 831) and any nucleic acids with the 2′-hydroxyl eliminated or modified.

Also preferred are “locked” nucleic acids (LNA) (Koshkin, A. A., et al, (1998) Tetrahedon 54, 3607 22-24), which contains a methylene linkage between the 2′ and the 4′ positions of the ribose. These modifications can increase stability to degradation by nucleases or improve thermal stability. Also included are nucleic acids that contain LNA, 2′-O-meRNA, or 2′-deoxy-2′-fluorouridine bases and also contain several consecutive DNA bases (i.e. if cleavage of RNA by RNAse H is desired).

DNA

Preferred nucleic acids also include all types of single stranded or double stranded DNA, and oligodeoxynucleotides. Preferred DNAs include any 5′ derivatized DNA, or 3′ derivatized DNA where the 5′ or 3′ ends have been capped, or labeled, or extended with additional nucleic acids, or amino acids, or a mutagen, or suitably derivatized in any way.

Sense and Antisense Nucleic Acids.

Also preferred are any antisense nucleic acids that include phosphodiester antisense oligonucleotides (ON) and antisense oligodeoxynucleotides (ODN).

Also preferred are any sense and/or antisense “backbone derivatized” oligonucleotides or “backbone derivatized” oligodeoxynucleotides where the sugar-phosphate “backbone” has been derivatized or replaced with “backbone analogues” which include phosphorothioate (PS), phosphorodithioate, phosphoroamidate, alkyl phosphotriester, or methylphosphonate linkages or other “backbone analogues”. Such “backbone derivatized” sense and/or antisense oligonucleotides or oligodeoxynucleotides include those with non-phosphorous backbone analogues such as sulfamate, 3′-thioformacetal, methylene(methylimino) (MMI), 3′-N-carbamate, or morpholino carbamate.

Mixed Backbone Nucleic Acid Derivatives.

In one type of backbone derivatized nucleic acids (sense and/or antisense), only one section of the sugar-phosphate backbone has been derivatized or replaced with backbone analogues. One example of a preferred ODN in this invention has the following sequence composition;

Phosphodiester Extension|Phosphorothioate G3139 antisense bcl2

5′-Amino-TT TTT TCT TTT TTT TCT CCC AGC GTG CGC
CAT-3′

Another class of “mixed” backbone derivatized nucleic acids (sense and/or antisense) is where the sugar-phosphate backbone has been derivatized or replaced with backbone analogues in an alternating or mixed fashion. For instance, the base sequence of a mixed backbone ON or mixed backbone ODN would be comprised of short sections (i.e. one, two or more bases) of phosphodiester linkages alternating with sections of one or more backbone analog linkages such as phosphorothioate, or phosphorodithioate, or phosphoroamidate, or alkyl phosphotriester, or methylphosphonate linkages. These linkages can be in any desirable order or ratio in order to obtain the desired characteristics such as solubility, hydrophobicity, charge, etc. Preferably, such mixed backbone nucleic acids would allow an optimal balance in lower toxicity with higher efficacy and stability.

Capped Nucleic Acids.

Also preferred are capped nucleic acids including phosphodiester antisense oligonucleotides, antisense ODNs and any sense or antisense backbone derivatized oligonucleotides or oligodeoxynucleotides where the 5′ or 3′ ends have been capped, or labeled, or extended with additional nucleic acids, or amino acids, or a mutagen.

Preferred examples of said capped antisense nucleic acids include 3′ capped oligonucleotides or oligodeoxynucleotides with hexylamine, 1,2-propanol, diethyleneglycol or 2,2-dimethyl-1,3-propanol coupled to their 3′ end, as disclosed by S. Dheur, et al, Antisense & Nucleic Acid Drug Dev. 9, 515-525 (1999), and references therein.

Hybrid Nucleic Acids.

Also preferred are any nucleic acid hybrids (i.e. RNA-DNA hybrids) including any sense or antisense “backbone derivatized” oligonucleotides or oligodeoxynucleotides where RNA and DNA are hybridized through complementary sequences to form double or triple strands. This includes any sense or antisense hybrids containing any type of 5′ derivatized RNA, or 3′ derivatized RNA, or 5′ derivatized DNA, or 3′ derivatized DNA where the 5′ or 3′ ends have been capped, or labeled, or extended with additional nucleic acids or amino acids, or suitably derivatized in any way.

Chimera Nucleic Acids.

Also preferred are nucleic acid chimeras (i.e. RNA-DNA chimeras) wherein the sense or antisense nucleic acid strand is comprised of one or more sections of RNA and one or more sections of DNA grafted together. Said nucleic acid chimeras include those containing amino acids, or a mutagen, or any suitable polymer (i.e. PEG) or is suitably derivatized in any way.

Some preferred examples of synthetic oligonucleotides and ODNs are disclosed by J. F. Milligan, et al., J. Medicinal Chem. 36(14): 1923-1937 (1993) and Y. Shoji et al., Antimicrob. Agents Chemotherapy, 40(7): 1670-1675 (1996).

Also included are synthetic nucleic acid polymers including sense and/or antisense peptide nucleic acids (PNA) disclosed by Egholm, et al, Nature 365: 566-568(1993) and references therein, including PNA clamps (Nucleic Acids Res. 23: 217(1995)) and peptide-PNA conjugates including those disclosed by M. R. Lewis, et al, Bioconj Chem. 13, 1176 (2002) and references therein.

Also preferred nucleic acids are nucleotide mimics or co-oligomers like phosphoric acid ester nucleic acids (PHONA), disclosed by Peyman, et al., Angew. Chem. Int. Ed. Engl. 36: 2809-2812 (1997). Also included are DNA and/or RNA, including any fragments or derivatives from viruses, bacteria, fungi and higher plants as well as from any tissue, cells, nuclei, chromosomes, cytoplasm, mitochondria, ribosomes, and other cellular sources.

Triplex-Forming Nucleic Acid.

A triplex-forming nucleic acid is a nucleic acid capable of forming a third, or triple strand with a specific DNA or RNA segment. Since the initial observation of triple-stranded DNA by Felsenfeld et al., J. Am. Chem. Soc. 79: 2023 (1957), oligonucleotide-directed triple helix formation has emerged as a valuable tool in molecular biology. Current knowledge suggests that triplex-forming nucleic acids can bind as third strands of DNA in a sequence specific manner in the major groove in homopurine/homopyrimidine stretches in duplex DNA. In one motif, a homopyrimidine oligonucleotide binds in a direction parallel to the purine strand in the duplex, as described by Moser and Dervan, Science 238: 645 (1987), Praseuth et al., Proc. Natl. Acad. Sci. USA 85: 1349 (1988), and Mergny et al., Biochemistry 30: 9791 (1991). In the alternate purine motif, a homopurine strand binds anti-parallel to the purine strand, as described by Beal and Dervan, Science 251: 1360 (1991). Also preferred are any triplex-forming PNAs and triplex-forming backbone derivatized nucleic acids defined herein.

Mutagenic Triplex-Forming Nucleic Acid.

A mutagen is any chemical capable of causing a mutation at the desired site of a double-stranded DNA molecule. Preferably the mutation restores the normal, functional sequence of the gene, inactivates an oncogene or activates an oncogene suppressor, or alters the function or inactivates a viral gene. Examples include radionuclides such as ¹²⁵I, ³⁵S and ³²P, and molecules become mutagenic with radiation, such as boron that interacts with neutron capture and iodine that interacts with auger electrons.

A mutagenic, triplex-forming nucleic acid is a mutagenic nucleic acid capable of forming a triple strand with a specific DNA or RNA segment and chemically modifying some portion of the segment Generally, a mutagenic nucleic acid hybridizes to a chosen site in the target gene, forming a triplex region, thereby bringing the attached mutagen into proximity with the target gene and causing a mutation at a specific site in the gene.

If the target gene contains a mutation that is the cause of a genetic disorder, then a mutagenic oligonucleotide is useful in this invention for mutagenic repair that may restore the DNA sequence of the target gene to normal. If the target gene is a viral gene needed for viral survival or reproduction or an oncogene causing unregulated proliferation, such as in a cancer cell, then the mutagenic oligonucleotide is useful in this invention for causing a mutation that inactivates the gene to incapacitate or prevent reproduction of the virus or to terminate or reduce the uncontrolled proliferation of the cancer cell. A mutagenic oligonucleotide is also a useful anti-cancer agent in this invention for activating a repressor gene that has lost its ability to repress proliferation.

Targeting or Biorecognition Molecules.

For the purposes of this invention, targeting or biorecognition molecules (moieties) are those that bind to a specific biological substance or site. The biological substance or site is considered the “target” of the biorecognition molecule or “targeting moiety” that binds to it. In the prior art, many drugs are “targeted” by coupling them to a targeting molecule that has a specific binding affinity for the cells, tissue or organism that the drug is intended for. For targeting a nucleic acid in this invention, a targeting molecule is coupled to a carrier substance that has a nucleic acid intercalator covalently coupled to it. Examples of targeting molecules useful in this invention are described below under “ligand” and “receptor”.

Ligand.

A ligand functions as a type of targeting or biorecognition molecule defined as a selectively bindable material that has a selective (or specific), affinity for another substance. The ligand is recognized and bound by a usually, but not necessarily, larger specific binding body or “binding partner”, or “receptor”. Examples of ligands suitable for targeting are antigens, haptens, biotin, biotin derivatives, lectins, galactosamine and fucosylamine moieties, receptors, substrates, coenzymes and cofactors among others.

When applied to this invention, a ligand includes an antigen or hapten that is capable of being bound by, or to, its corresponding antibody or fraction thereof. Also included are viral antigens, nucleocapsids and cell-binding viral derivatives including those from any DNA and RNA viruses, AIDS, HIV and hepatitis viruses, adenoviruses, adeno-associated viruses (AAV), alphaviruses, arenaviruses, coronaviruses, flaviviruses, herpesviruses, myxoviruses, oncornaviruses, papovaviruses, paramyxoviruses, parvoviruses, picornaviruses, poxviruses, reoviruses, rhabdoviruses, rhinoviruses, togaviruses and viroids; any bacterial antigens including those of gram-negative and gram-positive bacteria, acinetobacter, achromobacter, bacteroides, clostridium, chlamydia, enterobacteria, haemophilus, lactobacillus, neisseria, staphyloccus, and streptoccocus; any fungal antigens including those of aspergillus, candida, coccidiodes, mycoses, phycomycetes, and yeasts; any mycoplasma antigens; any rickettsial antigens; any protozoan antigens; any parasite antigens; any human antigens including those of blood cells, virus infected cells, genetic markers, heart diseases, oncoproteins, plasma proteins, complement factors, rheumatoid factors. Included are cancer and tumor antigens such as alpha-fetoproteins, prostate specific antigen (PSA) and CEA, cancer markers and oncoproteins, among others.

Other substances that can function as ligands for targeting are certain vitamins (i.e. folic acid, B₁₂), steroids, prostaglandins, carbohydrates, lipids, antibiotics, drugs, digoxins, pesticides, narcotics, neuro-transmitters, and substances used or modified such that they function as ligands.

Most preferred are certain proteins or protein fragments (i.e. hormones, toxins), and synthetic or natural polypeptides with cell surface affinity such as growth factors that include basic fibroblast growth factors (bFGF). Ligands also include various substances with selective affinity for receptors that are produced through recombinant DNA, genetic and molecular engineering. Except when stated otherwise, ligands of the instant invention also include the ligands as defined by K. E. Rubenstein, et al, U.S. Pat. No. 3,817,837 (1974).

Also included are monoclonal antibodies for targeting of peptide nucleic acid (PNA) or other nucleic acids (for example, W. M. Pardridge, et al (1995) Proc. Natl. Acad. Sci. U.S.A. 92, 5592.).

Also included are any suitable vitamins for targeting such as vitamin B6 (T. Zhu, et al., (1994) Bioconjugate Chem. 5, 312.). Also included are targeting receptors such as for liver cells using the asialo-glycoprotein receptors (X. M. Lu, et al, (1994) Nucl. Med. 35, 269). Also, included are suitable octreotides or octreotate, the carboxylic acid derivative of octreotide for targeting somatostatin receptors, among others. Also included are peptides which bind to integrins and the EGF receptor family.

Receptor.

A receptor functions as a type of targeting molecule defined for this invention as a specific binding body or “partner” or “ligator” that is usually, but not necessarily, larger than the ligand it can bind to. For the purposes of this invention, it is a specific substance or material or chemical or “reactant” that is capable of selective affinity binding with a specific ligand. A receptor can be a protein such as an antibody, a nonprotein binding body or a “specific reactor.”

When applied to this invention, a receptor includes an antibody, which is defined to include all classes of antibodies, monoclonal antibodies, chimeric antibodies, Fab fractions, fragments and derivatives thereof. Also included are antibodies used for specific cell or tissue targeting such as antibodies that bind to specific cell receptors such as anti-transferrin antibodies used to cross the blood brain barrier.

Under certain conditions, the instant invention is also applicable to using other substances as receptors. For instance, other receptors suitable for targeting include naturally occurring receptors, any hemagglutinins and cell membrane and nuclear derivatives that bind specifically to hormones, vitamins, drugs, antibiotics, cancer markers, genetic markers, viruses, and histocompatibility markers.

Other receptors also include enzymes, especially cell surface enzymes such as neuraminidases, plasma proteins, avidins, streptavidins, chalones, cavitands, thyroglobulin, intrinsic factor, globulins, chelators, surfactants, organometallic substances, staphylococcal protein A, protein G, ribosomes, bacteriophages, cytochromes, lectins, certain resins, and organic polymers.

Preferred targeting molecules also include various substances such as any proteins, protein fragments or polypeptides with affinity for the surface of any cells, tissues or microorganisms that are produced through recombinant DNA, genetic and molecular engineering.

Transduction Vector.

Transduction vectors are known in the prior art under a wide variety of names. For this invention a transduction vector is defined as a substance that promotes cellular uptake across the cell membrane and may include intracellular transport such as into the cell nucleus. Preferred transduction vectors or “fusion vectors” or “fusion moieties” or “membrane transduction” moieties include any suitable membrane translocation or membrane transfer substances that can include peptides, carbohydrates, lipids and polymers and combinations of these substances. Transduction vectors include proteins or peptides (“fusion peptides” or “peptide vectors”) including those with “transduction domains” in their amino acid sequence.

Some preferred transduction vectors for this invention include, but are not limited to any derived sequences or extracts of any signal peptides or any fusogenic peptides including: TAT (i.e. from HIV virus), herpes simplex virus VP-22, hepatitis B virus PreS2 translocation motif (TLM), antennapedia homeoproteins (i.e. penetratins). Also included are the peptide vectors disclosed by P. M. Fischer, et al, in Reviews Bioconj. Chem 12, 825-841 (2001) and references therein. Preferred examples of transduction vectors in this invention are peptide vectors which have been employed for nucleic acid transport into cells. Preferred examples include conjugates of a carrier substance with penetratins or signal peptides to increased uptake rates due to the membrane translocation properties of these peptides. Table I. is a list of some peptides that are preferred transduction vectors in this invention.

TABLE I

| | |
|---|---|
| TRANSDUCTION PEPTIDE SEQUENCE | NAME (origin of sequence) |

| | |
|---|---|
| RQIKIWFQNRRMKWKK | pAntp(43-58); Penetratin |
| KKWKMRRNQFWVKVQR | retro-inverso pAntp(43-58) |
| RRWRRWWRRWWRRWRR | W/R Penetratin |
| RQIKIWFQNRRMKWKKEN 24 | antennapedia peptide |
| RRMKWKK | pAntp(52-58) |
| GRKKRRQRRRPPQ | HIV TAT |
| YGRKKRRQRRR | HIV TAT |
| PTSQSRGDPTGPKE | HIV TAT C-terminus peptide |
| AVGAIGALFLGFLGAAG | viral fusion peptide |
| GALFLGWLGAAGSTMGA | gp41 fusion sequence |
| GALFLGFLGAAGSTMGAWSQPKSKRKV MPG | (gp41 fusion sequence SV40 NLS) |
| DRVIEVVQGAYRAIRNIPRRIRQG | CR-gp41 fusion peptide |
| MGLGLHLLVLAAALQGA | C. crocodylus Ig(v) light chain |
| MGLGLHLLVLAAALQGAWSQPKKKRKV | C. crocodylus Ig(v) light chain - SV40 NLS |
| PLSSIFSRIGDP | PreS2-TLM |
| GWTLNSAGYLLGKINLKALAALAKKIL | Transportan |
| RGGRLSYSRRRFSTSTGR | SynB1 |
| AAVALLPAVLLALLAP | MPS (kaposi FGF signal sequence) |
| AAVLLPVLLAAP | MPS (kaposi FGF signal sequence) |
| VTVLALGALAGVGVG | MPS (human integrin beta3 signal seq) |
| VAYISRGGVSTYYSDTVKGRFTRQKYNKRA | P3 |
| KLALKLALKALKAALKLA | Model amphiphilic peptide |
| WEAKLAKATAKALAKHLAKALAKALKACEA | KALA |
| GLFEAIAGFIENGWEGMIDGGGYC | hemagglutinin envelope fusion peptide |
| RRRRRRR | R7 |
| AAVALLPAVLLALLAPVQRKRQKLMP | engineered |
| MGLGLHLLVLAAALQGAKKKRKV | engineered |

Cell Receptor Binding Peptides.

Also preferred are known cell receptor binding peptides that bind to distinct receptors, which upon binding, mediate endocytosis of a peptide-ODN complex. Also included are peptides which bind to integrins and to the EGF receptor family. Table II. is a list of some receptor binding peptides that are preferred in this invention:

TABLE II

RECEPTOR BINDING

| | |
|---|---|
| PEPTIDE SEQUENCE | NAME (function) |

| | |
|---|---|
| TQPREEQYNSTFRV | Fc receptor binding peptide |
| D-GCSKAPKLPAALC | antagonist to IGF-1 receptor |
| YGGFLRRG | beta-endophin receptor ligand |
| YEE(ah-GalNAc)3 | hepatocyte specific delivery |
| Z-D-Phe-L-Phe-Gly | cell fusion and hemolysis |
| | inhibitor |

Amphiphilic Molecules.

Amphiphilic molecules are defined as those that contain at least one hydrophilic (polar) moiety and at least one hydrophobic (nonpolar) moiety. In certain embodiments of this invention, amphiphilic molecules including amphiphilic block polymers or copolymers are prepared for use as the carrier substance or as grafted polymers on the carrier substance. Most preferred are amphiphilic diblock or triblock copolymers prepared from a variety of monomers to provide at least one hydrophilic and one hydrophobic moiety. Amphiphilic cyclodextrin dimers, trimers and polymers as well as amphiphilic block copolymers containing CD dimers, trimers and polymers are included. Preferred amphiphilic molecules have a molecular weight range from 500 to 100,000 Daltons, preferably from 1,000 to 10,000 Daltons.

Amphiphilic molecules and copolymers can also introduce other desirable properties such as a positive or negative net charge. The desired targeting molecule or other substance can be coupled to available sites on the hydrophilic moieties of the amphiphilic molecule. Then, when the amphiphilic molecule is incorporated or “anchored” into a micelle with a nucleic acid carrier, the targeting molecule is thereby noncovalently coupled to the carrier of the instant invention.

Examples of suitable substances for use in amphiphilic molecules are certain proteins, polypeptides, polyamino acids, glycoproteins, lipoproteins (i.e. low density lipoprotein), amino sugars, glucosamines, polysaccharides, lipopolysaccharides, amino polysaccharides, polyglutamic acids, poly lactic acids (PLA), polylysines, polyethylenimines, polyacrylamides, nylons, poly(allylamines), lipids, glycolipids and suitable synthetic polymers, especially biopolymers, resins and surfactants, as well as suitable derivatives of these substances. Also included as suitable substances are the polymers disclosed in U.S. Pat. No. 4,645,646. Also preferred for use in amphiphilic molecules are N-(2-hydroxypropyl) methacrylamide (HPMA), HPMA derivatives, poly cyanoacrylates such as poly(butyl cyanoacrylate), poly(isobutyl or isohexyl cyanoacrylate), polyethylene glycol (PEG), any micelle-forming PEG derivatives, poly (D,L-lactic-coglycolic acid) (PLGA), PLGA derivatives and poly (D,L-lactide)-block-methoxypolyethylene glycol (diblock).

Also included are any micelle-forming copolymers that contain poly(ethylene oxide) (PEO) such as PEO-block-poly(L lysine), PEO-block-poly(aspartate), PEO-block-poly(beta-benzyl aspartate), PEO-block-poly(lactic acid), PEO-block-poly(L-lactic-coglycolic acid), PEO-block-poly(propylene oxide) (PPO) and any derivatives. Also preferred are any micelle-forming triblock copolymers (Pluronics) that contain PEO and polypropylene oxide) (PPO), such as PEO-block-PPO-block-PEO in various ratios. Specific examples are the F, L or P series of Pluronics including F-68, F-108, F-127, L-61, L-121, P-85, and any derivatives.

With suitable modification of the synthesis methods referenced by G. S. Kwon, IN: Critical Reviews in Therapeutic drug Carrier Systems, 15(5): 481-512 (1998), suitable grafted polymers as described herein or amphiphilic molecules can be synthesized for preparing the nucleic acid carriers of this invention. Included are diblock and triblock copolymer synthesis methods include ring-opening polymerization such as with PEO and various N carboxyanhydride (NCA) monomers; polymerizations using triphosgenes and organo-metal (i.e. nickel) initiators (i.e. stannous octoate). Also useful are anionic, zwitterionic and free radical polymerizations and transesterifications, among others.

Cyclodextrin.

A cyclodextrin (CD) monomer, is an oligosaccharide composed of glucose molecules coupled together to form a ring that is conical with a hydrophobic, hollow interior or cavity. Cyclodextrin monomers are one of the starting materials for making grafted polymers as described in the instant invention. They can be any suitable cyclodextrin, including alpha-, beta-, and gamma-cyclodextrins, and their combinations, analogs, isomers, and derivatives.

In describing this invention, references to a cyclodextrin “complex”, means a noncovalent inclusion complex. An inclusion complex is defined herein as a cyclodextrin functioning as a “host” molecule, combined with one or more “guest” molecules that are contained or bound, wholly or partially, within the hydrophobic cavity of the cyclodextrin or its derivative.

Cyclodextrin Dimers, Trimers and Polymers.

For this invention, a cyclodextrin dimer is a preferred type of cyclodextrin derivative defined as two cyclodextrin molecules covalently coupled or cross-linked together to enable cooperative complexing with a guest molecule. Examples of some CD dimers that can be derivatized and used in the drug carriers of this invention, are described by; Breslow, R., et al, Amer. Chem. Soc. 111, 8296-8297 (1989); Breslow, R., et al, Amer. Chem. Soc. 105, 1390 (1983) and Fujita, K., et al, J. Chem. Soc., Chem. Commun., 1277 (1984).

A cyclodextrin trimer is another preferred type of cyclodextrin derivative defined as three cyclodextrin molecules covalently coupled or cross-linked together to enable cooperative complexing with a guest molecule. Another preferred cyclodextrin is a cyclodextrin polymer defined as a unit of more than three cyclodextrin molecules covalently coupled or cross-linked together to enable cooperative complexing with several guest molecules. Also included are the “linear” cyclodextrin polymers disclosed by Davis, et al, U.S. Pat. No. 6,509,323 B1.

For this invention, preferred cyclodextrin dimer, trimer and polymer units are synthesized by covalently coupling through chemical groups such as through coupling agents. The synthesis of preferred cyclodextrin dimer, trimer and polymer units does not include the use of proteins or other “intermediate coupling substances”. Cooperative complexing means that in situations where the guest molecule is large enough, the member cyclodextrins of the CD dimer, trimer or polymer can each noncovalently complex with different parts of the same guest molecule, or with smaller guests, alternately complex with the same guest.

The prior art has disclosed dimers and polymers comprised of cyclodextrins of the same size. An improved cyclodextrin dimer, trimer or polymer comprises combinations of different sized cyclodextrins to synthesize these units. These combinations may more effectively complex with guest molecules that have heterogeneous complexing sites. Combinations for this invention can include the covalent coupling of an alpha CD with a beta CD, an alpha CD with a gamma CD, a beta CD with a gamma CD and polymers with various ratios of alpha, beta and gamma cyclodextrins.

Most preferred are cyclodextrin dimers, trimers and polymers containing cyclodextrin derivatives such as carboxymethyl CD, glucosyl CD, maltosyl CD, hydroxypropyl cyclodextrins (HPCD), 2-hydroxypropyl cyclodextrins, 2,3-dihydroxypropyl cyclodextrins (DHPCD), sulfobutylether cyclodextrins (SBECD), ethylated and methylated cyclodextrins.

Also preferred are oxidized cyclodextrin dimers, trimers and polymers that provide aldehydes and any oxidized derivatives that provide aldehydes. Some examples of suitable derivatives are disclosed by Pitha, J., et al, J. Pharm. Sci. 75,165-167 (1986) and Pitha, J., et al, Int. J.

Pharmaceut. 29, 73-82 (1986).

Also preferred are any amphiphilic CD dimers, trimers and polymers made from derivatives such as those disclosed by K. Chmurski, et al., Langmuir 12, 4046 (1996), P. Zhang, et al., J. Phys. Org. Chem. 5, 518 (1992), M. Weisser, et al., J. Phys. Chem. 100, 17893 (1996), L. A. Godinez, et al., Langmuir 14, 137 (1998) and D. Duchene, “International Pharmaceut. Applic. of Cyclodextrins Conference”, Lawrence, Kans., USA, Jun. 1997, and references therein.

Also included are altered forms, such as crown ether-like compounds prepared by Kandra, L., et al, J. Inclus. Phenom. 2, 869-875 (1984), and higher homologues of cyclodextrins, such as those prepared by Pulley, et al, Biochem. Biophys. Res. Comm. 5, 11 (1961). Some useful reviews on cyclodextrins are: Atwood J. E. D., et al, Eds., “Inclusion Compounds”, vols. 2 & 3, Academic Press, NY (1984); Bender, M. L., et al, “Cyclodextrin Chemistry”, Springer-Verlag, Berlin, (1978) and Szejtli, J., “Cyclodextrins and Their Inclusion Complexes”, Akademiai Kiado, Budapest, Hungary (1982). These references, including references contained therein, are applicable to the synthesis of the preparations and components of the instant invention and are hereby incorporated herein by reference.

Cyclodextrin Blocks.

A CD-block is defined as a CD dimer, trimer or polymer that is used as a component, or unit (i.e. building block) for additional cross linking with other polymer blocks to produce a carrier substance or are coupled to the carrier substance of this invention.

Preferred cyclodextrin blocks (CD block) are compositions that provide for the incorporation of cyclodextrin derivatives into carrier substances that include micelle-forming amphiphilic molecules through copolymerization with other polymer blocks or grafted polymers defined herein. The CD blocks can include CD dimers, CD trimers or CD polymers. The CD blocks can be primarily hydrophilic to produce micelles with the CD moieties in the hydrophilic shell. Or, the CD blocks can be primarily hydrophobic to produce micelles with the CD moieties in the hydrophobic core.

The CD blocks also have available suitable reactive groups that can copolymerize with other block polymers, using suitably modified methods described and referenced by G. S. Kwon, IN: Critical Reviews in Therapeutic drug Carrier Systems, 15(5): 481-512 (1998).

For example, a CD derivative (i.e. CD dimer) is prepared and made hydrophobic by adding alkyl or aromatic groups (i.e. methylation, ethylation, or benzylation), and also has available an N carboxyanhydride (NCA) group coupled through a suitable spacer.

One form of CD block would be methylated-CD-CD-poly(aspartate)_{N}-NCA (where N=1-10). This CD block can then be copolymerized with suitable blocks of alpha-methyl-omega-amino-poly(ethylene oxide) (PEO) in suitable solvent (CHCl₃:DMF) to produce a micelle-forming diblock amphiphilic molecule. The resulting diblock is CD-block-PEO. With suitable modifications PEG can be used in place of PEO. Also, triblocks such as PEO-block-CD-block-PEO can be prepared.

Other combinations for the CD-blocks of this invention can include the covalent coupling of an alpha CD with a beta CD, an alpha CD with a gamma CD, a beta CD with a gamma CD and polymers with various ratios of alpha, beta and gamma cyclodextrins.

Grafted Polymer.

A grafted polymer is defined as any polymeric substances including copolymers and block polymers that are suitably coupled to produce a carrier substance as defined in the present invention. Preferably grafted polymers are biocompatible and generally hydrophilic. Preferred grafted polymers include polyethylene glycols (PEG), methoxy polyethylene glycols (mPEG), N-(2-hydroxypropyl) methacrylamide polymer (HPMA), poly(2-(dimethylamino) ethyl methacrylate (DMAEMA), poly(lactide-co-glycolide) (PLGA), poly(polypropyl acrylic acid) (PPAA), polyethylenimine (PEI), polyamidoamines (PAMAM), polylysine (PLL), CD dimers, CD trimers, CD polymers and CD blocks, defined herein. Preferably grafted polymers also include any suitable combination of the polymers defined herein. Wherein said grafted polymer is appropriately endcapped as is known in the prior art and which also may be substituted with moieties that do not adversely affect the functionality of the grafted polymer for its intended purpose. The CD-grafted polymers of the present invention can be synthesized by coupling two to thirty CDs or derivatives thereof to a carrier substance.

Liposome.

A liposome or vesicle is defined as a water soluble or colloidal structure composed of amphiphilic molecules that have formed generally spherical bilayer membranes. Said amphiphilic molecules are generally oriented in said bilayer membrane so that their hydrophilic ends are on the outside of the membrane and their hydrophobic ends are sequestered inside the membrane. Preferred liposomes of this invention generally have a spherical shape where said bilayer membranes are arranged in one or more layers (lamella) around a single, primarily hydrophilic or aqueous, central zone. Unilamellar liposomes have one bilayer membrane surrounding a central hydrophilic zone. Multilamellar liposomes have more than one surrounding membrane with hydrophilic zones between said membranes that surround the central hydrophilic zone.

Liposomes can be composed of any suitable amphiphilic molecules described herein. Also, the amphiphilic molecules can be suitably polymerized or cross linked, including the use of biocleavable linkages.

Micelles and Nanoparticles.

A preferred micelle or nanoparticle for this invention is defined as a water soluble or colloidal structure or aggregate (also called a nanosphere) composed of one or more amphiphilic molecules and may include grafted polymers defined herein. Preferred micelles and nanoparticles of this invention generally have a single, central and primarily hydrophobic zone or “core” surrounded by a hydrophilic layer or “shell”. This shape may also be due to aggregation and/or condensation of the carrier due to self attraction.

Preferred micelles for use as carrier substances in this invention include those disclosed in U.S. patent application Ser. No. 09/829,551, filed Apr. 10, 2001, the contents of which are incorporated herein.

Also preferred are nanoparticles composed of macromolecules including “cascade polymers” such as dendrimers. Preferred dendrimers include polyamidoamines as disclosed by J. Haensler, et al, in Bioconj. Chem. 4, 372-379 (1993).

Micelles and nanoparticles range in size from 5 to about 2000 nanometers, preferably from 10 to 400 nm. Micelles and nanoparticles of this invention are distinguished from and exclude liposomes which are composed of bilayers.

The micelles of this invention can be composed of either a single monomolecular polymer containing hydrophobic and hydrophilic moieties or an aggregate mixture containing many amphiphilic (i.e. surfactant) molecules formed at or above the critical micelle concentration (CMC), in a polar (i.e. aqueous) solution.

Pendant PEG.

Pendant polyethylene glycol is one preferred carrier substance for synthesizing the compositions of the present invention. Pendant PEG is defined here as derivatized or “grafted” with side functional groups or “branches” along the backbone of the molecule. The functional groups are frequently propionic acid groups comprising a three carbon alkyl side chain with a terminal carboxylic acid. However, the grafted functional side group can be comprised of alkyl chains of 2, 3, 4, 5, 6, or more carbon atoms that terminate in carboxylic acid, or a primary amine, or an aldehyde, or a thiol, or combinations of these.

Pendant PEG (also called “multi-branched PEG”), is commercially available in a variety of molecular masses and with various numbers of functional groups per molecule. For instance, SunBio USA, Orinda, Calif. 94563, offers such material in molecular weights of 10, 12, 18, 20, 30, 35 and 100 kilo Daltons (KDa) and with 6, 8, 10, 12, 14, 16, 18, or 20 functional side groups or “branches” per molecule.

For the present invention, preferred pendant PEG starting material ranges from 6,000 Daltons to 100,000 Daltons, most preferably a molecular weight of 20,000 or greater to prevent rapid elimination of the PEG-conjugated composition from the bloodstream.

In one preferred embodiment of the present invention, a carboxyl group grafted PEG (20,000 Daltons or 25,000 Daltons containing 8 to 15 carboxyl groups per PEG molecule) is used as the starting material to conjugate with the nucleic acids. In order to keep the steric hindrance effect to a minimum, a flexible linear linkage may be used to keep the nucleic acid moiety away from the polymer backbone. Due to the biocompatibility of the materials and pliability of the polymers of the present invention, they will cause minimal toxicity.

Targeted Nucleic Acid Carriers.

A targeted nucleic acid carrier is composed of a nucleic acid carrier substance that has a targeting molecule covalently coupled to it. The carrier is thereby targeted through the specific binding properties of the targeting molecule coupled to the surface. During the coupling of the targeting molecule, the functions of the targeting molecule and the targeted carrier are not irreversibly or adversely inhibited. Preferably, the targeting molecule maintains specific binding properties that are functionally identical or homologous to those it had before coupling. Preferably, the targeting molecule is coupled through a suitable spacer to avoid steric hindrance.

Targeted carriers coupled to avidin and streptavidin are useful for subsequent noncovalent coupling to any suitable biotinylated substance. Similarly, nucleic acid carriers coupled to antibody can be noncovalently coupled to another antibody, or to a peptide or other suitable substance that has the appropriate biorecognition properties. Another useful nucleic acid carrier comprises protein A, protein G, or any suitable lectin or polypeptide that has been covalently coupled to a nucleic acid carrier of this invention.

Capping Moiety.

A capping moiety is defined here as a substance that is used to consume or cap any available reactive groups or functional groups to prevent further coupling or other reactions on the carrier of this invention. The capping moiety may also provide certain desired properties such as neutral charge, or positive charge or negative charge as desired. The capping moiety may also provide increased water solubility or may provide hydrophobicity. The capping moiety may also provide a type of label for colorimetric or fluorometric detection.

Some preferred examples of capping moieties are ethanol amines, glucose amines, mercaptoethanol, any suitable amino acids, including gylcines, alanines, leucines, phenylalanines, serines, tyrosines, tryptophanes, asparagines, glutamic acids, cysteines, lysines, arginines and histidines, among others. Preferred capping moieties also include suitable fluorophores or dyes.

Pendant Polyethylene Glycol Nucleic Acid Carrier.

One preferred “unloaded” nucleic acid carrier is defined as a pendant PEG polymer backbone wherein intercalator moieties are covalently coupled to said pendant PEG through branched functionalities on the backbone. Nucleic acids are “loaded” onto the carrier by coupling them to the carrier through intercalation with said intercalators. The polymeric composition of this invention is a mixture of polymer units where the number of units in the polymer may be variable and the number of intercalator moieties may vary. Hence, each polymer has an average molecular weight and an average number of intercalators per polymer backbone within such polymeric composition. Said pendant polyethylene glycol polymer backbone has a molecular weight range from 2,000 to 1,000,000 Daltons, preferably 5,000 to 70,000 Daltons, and most preferably 20,000 to 40,000 Daltons.

Accordingly, the unloaded, pendant polyethylene glycol carrier of the present invention can be represented by the following formula:

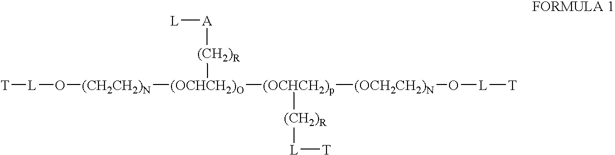

Formula 1 shows a horizontal polyethylene glycol backbone comprising connected units; (OCH₂CH₂)N , (OCHCH₂)_{O} and (OCHCH₂)_{P}; which may alternate in their number, sequence and frequency within the polymer backbone.

For said connected units, N and O are independent integers equaling average values between 1 and 30 of their respective units. P is an independent integer equaling an average value between 0 (zero) and 30 of its respective unit, meaning that if P=zero there are no respective units for P in the backbone. Also, wherein a mixture of different N, O and P values may be found for their respective units.

The polymer backbone also includes the branching or pendant unit; (CH₂)_{R} covalently coupled to said PEG backbone and wherein R is an integer between 1 and 30, preferably between 2 and 10. Also, wherein said pendant unit terminates in either a functional group or is terminally coupled to moieties “L-A” or “L-T” as defined below.

In Formula 1, A is an intercalator as disclosed herein independently and covalently coupled to the pendant polyethylene glycol backbone through linkage L.

In Formula 1, T is independently and covalently coupled to the pendant polyethylene glycol backbone through linkage L.

T is a member independently selected from the group consisting of hydrogen (H), or hydroxyl (OH), or a targeting moiety (TM), or a transduction vector (TV), amphiphilic molecule, or a capping moiety.

T may also be a member independently selected from the group consisting of a grafted polymer as disclosed herein that is biocompatible and includes PEG, HPMA, PEI, PLL, CD, CD dimers, CD trimers and CD polymers. Wherein said grafted polymer is appropriately endcapped as is known in the prior art and which also may be substituted with substituents that do not adversely affect the functionality of the grafted polymer for its intended purpose.

Also wherein said grafted polymer has a molecular weight range from 500 to 100,000 Daltons, preferably from 1,000 to 10,000 Daltons.

Also wherein T as described herein is coupled to said pendant polyethylene glycol backbone with the proviso that a mixture of hydrogen, hydroxyl, targeting moieties, cell transduction vectors, amphiphilic molecule and grafted polymer may be found on the same polyethylene glycol backbone and/or within the same polyethylene glycol polymer composition.

L is a covalent linkage between said polyethylene glycol and nucleic acid intercalator A or T as defined herein, through functional groups defined herein and may include one or more coupling agents as defined herein. Said linkage L may also include suitable spacer molecules and may be biocleavable as defined herein.

### EXAMPLES OF THE BEST MODES FOR CARRYING OUT THE INVENTION

In the examples to follow, percentages are by weight unless indicated otherwise. During the synthesis of the compositions of the instant invention, it will be understood by those skilled in the art of organic synthesis, that there are certain limitations and conditions as to what compositions will comprise a suitable polymer carrier and may therefore be prepared mutatis mutandis. It will also be understood in the art of nucleic acids that there are limitations as to which derivatives and/or coupling agents can be used with nucleic acids to fulfill their intended function.

The terms “suitable” and “appropriate” refer to synthesis methods known to those skilled in the art that are needed to perform the described reaction or procedure. In the references to follow, the methods are hereby incorporated herein by reference. For example, organic synthesis reactions, including cited references therein, that can be useful in the instant invention are described in “The Merck Index”, 9, pages ONR-1 to ONR-98, Merck & Co., Rahway, N.J. (1976), and suitable protective methods are described by T. W. Greene, “Protective Groups in Organic Synthesis”, Wiley-Interscience, NY (1981), among others. For synthesis of nucleic acid probes, sequencing and hybridization methods, see “Molecular Cloning”, 2nd edition, T. Maniatis, et al, Eds., Cold Spring Harbor Lab., Cold Spring Harbor, N.Y. (1989).

All reagents and substances listed, unless noted otherwise, are commercially available from Aldrich Chemical Co., WI 53233; Sigma Chemical Co., Mo. 63178; Pierce Chemical Co., IL. 61105; Eastman Kodak Co., Rochester, N.Y.; Pharmatec Inc., Alachua Fla. 32615; and Research Organics, Cleveland, Ohio. Or, the substances are available or can be synthesized through referenced methods, including “The Merck Index”, 9, Merck & Co., Rahway, N.J. (1976). Additional references cited in U.S. Pat. No. 6,048,736 and PCT/US99/30820, are hereby incorporated herein by reference.

### Nucleic Acid Carriers

The general synthesis approach is; (1) produce or modify or protect, as needed, one or more functional groups on the carrier substance and (2) using one or more coupling methods, covalently couple a nucleic acid intercalator to the carrier substance.

Also, as described below, the carrier may be suitably derivatized to include other useful substances and/or chemical groups (e.g. targeting molecules), to perform a particular function. Depending on the requirements for chemical synthesis, the derivatization can be done before coupling the intercalator, or afterward, using suitable protection and deprotection methods as needed.

The carrier substance can be suitably derivatized and coupled through well-known procedures used for available amino, sulfhydryl or hydroxyl groups. Also, for certain carbohydrates added to the carrier substance, vicinal hydroxyl groups can be appropriately oxidized to produce aldehydes. Any functional group can be suitably added through well-known methods while preserving the carrier substance structure and properties. Examples are: amidation, esterification, acylation, N-alkylation, allylation, ethynylation, oxidation, halogenation, hydrolysis, reactions with anhydrides, or hydrazines and other amines, including the formation of acetals, aldehydes, amides, imides, carbonyls, esters, isopropylidenes, nitrenes, osazones, oximes, propargyls, sulfonates, sulfonyls, sulfonamides, nitrates, carbonates, metal salts, hydrazones, glycosones, mercaptals, and suitable combinations of these. The functional groups are then available for the cross-linking using a bifunctional reagent.

Suitable coupling or cross-linking agents for preparing the carriers of the instant invention can be a variety of coupling reagents, including oxiranes and epoxides previously described. Also useful are methods employing acrylic esters such as m-nitrophenyl acrylates, and hexamethylene diamine and p-xylylenediamine complexes, and aldehydes, ketones, alkyl halides, acyl halides, silicon halides and isothiocyanates.

Synthesis of Nucleic Acids With Suitable Functional Groups.

Because conventional automated synthesis of nucleic acids proceeds from 3′ to 5′, the 5′-terminus is readily available for the addition of functional groups. A general approach to the modification of the 5′-terminus is to use reagents which couple to the 5′-hydroxyl of an oligonucleotide. In this invention, the phosphoramidite reagents used include those that are compatible with automated DNA synthesizers. These reagents are available from Glen Research Corp., Sterling, Va., and other suppliers. 5′-Modified Nucleic Acids.

A preferred group of phosphoramidite reagents is the 5′-Amino-Modifiers. The 5′-Amino-Modifiers are preferably for use in automated synthesizers to functionalize the 5′-terminus of a target oligonucleotide. The primary amine can be used to attach a variety of functional moieties to the oligonucleotide.

The 5′-Amino-Modifiers include 6-(4-Monomethoxytritylamino) hexyl-(2-cyanoethyl)-(N,N-diisopropyl)-phosphoramidite, M.W.: 589.76; 12-(4-Monomethoxytritylamino) dodecyl-(2-cyanoethyl)-(N,N-diisopropyl)-phosphoramidite, M.W.: 673.92 and 2-[2-(4-Monomethoxytrityl) aminoethoxy]ethyl-(2-cyanoethyl)-(N,N-diisopropyl)-phosphoramidite, M.W.: 577.71.

Also included are 6-(Trifluoroacetylamino)propyl-(2-cyanoethyl)-(N,N-diisopropyl)-phosphoramidite, M.W.: 371.34 and 6-(Trifluoroacetylamino)hexyl-(2-cyanoethyl)-(N,N-diisopropyl)-phosphoramidite, M.W.: 413.42.

Another group of preferred reagents for adding an amino group are 5′-amino-modifiers such as β-cyanoethyl (CE) phosphoramidites which, when activated with 1H-tetrazole, can couple to the 5′-terminus of the nucleic acid with similar efficiency as nucleoside phosphoramidites.

5′-Thiol Nucleic Acid.

The phosphorothioate nucleic acids are synthesized using beta-cyanoethyl phosphoramidite chemistry. Acetylation is performed by 0.1 M acetic anhydride/tetrahydrofuran (THF) and 0.1 M imidazole/THF. Sulfurization is done by using the EDITH reagent.

The commercially available six-carbon thiol linker phosphoramidite (1-O-dimethoxytrityl-hexyl-disulfide-1′-[(2-cyanoethyl)-(N,N-diisopropyl)]-phosphoramidite (Glen Research) is coupled to the 5′ end. The final coupling is followed by an acetonitrile wash. The resin is dried under a stream of argon and treated with concentrated ammonia containing 0.1 M DTT at 55° C. for 12 h to simultaneously affect deprotection of the thiol protection as well as cleavage from the resin. The resin is removed by filtration and rinsed with concentrated ammonia. Evaporation of the resultant solution affords a clear residue which is dissolved in sterile water. To remove excess DTT, the solution is passed through a NAP-10 gel filtration column. The fractions containing the nucleic acid are immediately used for conjugation to the carrier.

Another preferred phosphoramidite reagent for adding a thiol functional group includes (S-Trityl-6-mercaptohexyl)-(2-cyanoethyl)-(N,N-diisopropyl)-Phosphoramidite M.W.: 576.78, which produces a thiol group at the 5′-terminus of a synthetic oligonucleotide or nucleic acid. Alternatively, coupling to the 3′-terminus, it is added to any suitable support and then the desired nucleic acid is synthesized. DTT is used during deprotection or after purification of the product nucleic acid to cleave the disulfide linkage.

A. Kumar, et al, in Nucleic Acids Res., 19, 4561 (1991) describes a procedure useful in this invention to modify a 5′-amino-modified oligonucleotide to a thiol using N-acetyl-DL-homocystein thiolactone. Another preferred group includes those designed to introduce a thiol group to the 3′-terminus of a target oligonucleotide such as 1-O-Dimethoxytrityl-propyl-disulfide, 1′-succinoyl-long chain alkylamino-CPG.

Another preferred group of phosphoramidite reagents in this invention includes spacer phosphoramidites such as 9-O-Dimethoxytrityl-triethyleneglycol, 1-[(2-cyanoethyl)-(N,N-diisopropyl)]-phosphorarmidite, M.W.: 652.77, 18-O-Dimethoxytrityl-hexaethyleneglycol, 1-[(2-cyanoethyl)-(N,N-diisopropyl)]-phosphoramidite, M.W.: 784.93, 3-O-Dimethoxytrityl-propyl-1-[(2-cyanoethyl)-(N,Ndiisopropyl)]-Phosphoramidite, M.W.: 578.69, 12-O-Dimethoxytrityl-dodecyl-1-[(2-cyanoethyl)-(N,Ndiisopropyl)]-phosphoramidite, M.W.: 704.93 and 5′-O-Dimethoxytrityl-1′,2′-Dideoxyribose-3′-[(2-cyanoethyl)-(N,N-diisopropyl)]-phosphoramidite, M.W.: 620.73.

In this invention, the spacer phosphoramidites can be used to insert a mixed polarity 9 or 18 atom spacer arm in a nucleic acid. These compounds may be added in multiple additions when a longer spacer is required. The spacer phosphoramidites can also be added to substitute for bases within a nucleic acid sequence and to mimic an abasic site in an oligonucleotide.

Colored or Fluorescent Labeling.

Another preferred group of phosphoramidite reagents useful in this invention is any suitable colored or fluorescent labeling moiety. This includes any suitable 3′ or 5′-labelling reagent. Fluorescent derivatives are useful in tracking nucleic acids and/or the carrier in vivo or in vitro. Included are any fluorescein derivatives (i.e. 6-FAM, HEX and TET, derived from the 6-carboxy fluorescein isomer). Also included are any cyanine dye derivatives (i.e. Cy3 and Cy5 phosphoramidites) and phosphoramidite reagents with dabcyl or TAMRA labels.

Another preferred group of phosphoramidite reagents useful in this invention includes any suitable 3′ or 5′-Biotin phosphoramidite reagent for adding biotin to the nucleic acid to provide a specific coupling site with any suitable avidin or streptavidin. Biotin labeling phosphoramidites are capable of branching to allow multiple biotins to be introduced at the 3′- or 5′-terminus while biotin-dT can replace dT residues within the oligonucleotide sequence.

3′-Modified Nucleic Acids.

In the design and synthesis of antisense nucleic acids in this invention, there are preferred reagents for use in modifying the 3′-terminus of oligonucleotides. This may be achieved by modifying the 3′-terminus with a phosphate group, a phosphate ester, or using an inverted 3′-3′ linkage. Nucleic acids modified at the 3′-terminus resist 3′-exonuclease digestion and thereby provide a more effective agent in vivo.

A preferred group of phosphoramidite chemical reagents for 3′ phosphorylation includes 2-[2-(4,4′-Dimethoxytrityloxy)ethylsulfonyl]ethyl-(2-cyanoethyl)-(N,N-diisopropyl)-phosphoramidite, among others.

A preferred simpler process is to couple any suitable phosphoramidite reagent that is desired for modifying the 3′ end of a nucleic acid onto the support such as controlled pore glass (CPG). The coupled phosphoramidite is used as the starting compound for synthesizing the nucleic acid. Said coupling is designed for subsequent cleavage using suitable chemical methods to provide the desired 3′ modification.

A preferred method is described by H. Urata, et al, Tetrahedron Lett., 34, 4015-4018 (1993) for the preparation of oligonucleotides with a 3′-phosphoglyceryl terminus. The terminus is readily oxidized by sodium periodate to form a 3′-phosphoglycaldehyde. The aldehyde may be further oxidized to the corresponding carboxylic acid. Either the aldehyde or the carboxylate may be used for subsequent conjugation to amine-containing moieties.

Another preferred embodiment in synthesizing the compositions of this invention is to couple sense or antisense nucleic acids through the 3′-terminus. One preferred approach to 3′-modification is to prepare said nucleic acid with a ribonucleoside (RNA) terminus, (i.e. nucleic acid chimera) using an RNA support. Subsequent oxidation of the 2′,3′-diol cleaves the 2′-3′ bond and generates reactive aldehyde groups. The resulting 3′ aldehyde group is then available for specific coupling to the carriers of this invention. The nucleic acid methods and references disclosed within the following references are hereby incorporated into this invention.
1. B. A. Connolly, et al, Nucleic Acids Res., 1985, 13, 4485.
2. N. G. Dolinnaya, et al, Nucleic Acids Res., 1993, 21, 5403-5407.
3. G. B. Dreyer, et al, Proc. Natl. Acad. Sci. USA, 1985, 82, 968.
4. M. Durard, et al, Nucleic Acids Res., 1990, 18, 6353.
5. M. W. Kalnik, et al, Biochemistry, 1988, 27, 924-931.
6. M. Lemaitre, et al, Proc. Nat. Acad. Sci. USA, 1987, 84, 648.
7. M. Lemaitre, et al Nucleosides & Nucleotides, 1987, 6, 311.
8. P. Li, et al., Nucleic Acids Res., 1987, 15, 5275.
9. M. Salunkhe, et al, J. Amer. Chem. Soc., 1992, 114, 8768-8772.
10. B. S. Sproat, et al, Nucleic Acids Res., 1987, 15, 4837.
11. M. Takeshita, et al. J. Biol. Chem., 1987, 262, 10171-10179.
12. R. Zuckerman, et al, Nucleic Acids Res., 1987, 15, 5305.

Synthesis Materials.

All chemicals were reagent grade and are available from Alltech Assoc., Deerfield, Ill., Amersham Pharmacia Biotech, Piscataway, N. J., Calbiochem, San Diego, Calif., Molecular Probes, Eugene, Oreg., Promega Corp., Madison, Wis., VWR International., West Chester, Pa. 19380, or Sigma-Aldrich, St. Louis, Mo. 63178. Deionized water is used where not stated otherwise. Some reagents used and their abbreviations are; 1-Decene, n-butylamine, 2,2,2-trifluoroethanol, 3-nitrophenol, fluorescein isothiocyanate (FITC), N-hydroxysuccinimide (NHS), ethanethiol, n-butylamine, 4-(dimethylamino)-pyridine (DMAP), dithiothreitol (DT), 1,1,2-trichloroethane (TCE), sodium dodecyl sulfate (SDS) and 1,3-diisopropylcarbodimide (DIC). Some solvents used are ethyl acetate (EtOAc), tetrahydrofuran (THF), and n-Heptane, N,N-dimethylformamide (DMF). Phosphate-buffered saline (PBS) is 0.01 M sodium phosphate and 0.015 M sodium chloride, pH about 7.2 or adjusted with 0.1 M HCl, 0.1 M KOH (or NaOH) solution as needed.

Testing Procedures.

The psoralen or trioxsalen concentration in the preparations was determined by fluorescence at 340 nm excitation wavelength and 528 nm emission wavelength. The sample concentration was determined by using least squares (linear regression) calculation of the slope and intercept from a standard curve of known concentrations.

Aldehyde concentration in the preparations was determined using the fluorescent indicator, 4′-Hydrazino-2-Stilbazole Dichloride (HSD) based on the method of S. Mizutani, et al, in Chem.

Pharm. Bull. 17, 2340-2348 (1969). The sample concentration was determined by using least squares calculation as described previously.

Amine concentration was measured by the following colorimetric test. To 0.02 mL of amine sample in water was added 0.2 mL of borate buffer, pH 8.5. Then 0.05 mL of 0.075% 2,4,6-trinitrobenzene sulfonate (TNBS) was added and mixed. After 20 minutes at rt, the absorbance was read at 525 nm. The absorbance was compared to a glycine standard curve to calculate the sample amine concentration by least squares as described previously.

Carbohydrate concentration was measured by the following colorimetric test. To 0.02 mL of carbohydrate sample in water was added 0.01 mL of 1.5% naphthol in MetOH. Then 0.1 mL of concentrated sulfuric acid was added rapidly to mix. After 20 minutes at rt, the absorbance was read at 620 nm. The absorbance was compared to a dextran or CD standard curve to calculate the sample concentration by least squares as described previously.

Thiol concentration was measured by combining: 0.008 ml of sample and 0.1 ml of 0.0125% 2,2′-dithio-bis(5-nitropyridine) (DTNP) in 62.5% isopropanol, pH 6 to produce a color reaction. The absorbance was read at 405 nm and sample thiol concentration was calculated by linear regression using values from a cysteine standard curve.

### Coupling Methods Using Activated Esters

The following are methods for synthesizing the carrier compositions of this invention. They are based on J. T. C. Wojtyk, et al, in Langmuir 18, 6081 (2002), for derivatizing a carboxylate group on any suitable carrier substance to provide an activated ester for coupling to a primary amine on an intercalator or any suitable substance.

If needed, a carrier substance with a hydroxyl or amino group such as protein, dextran, cyclodextrin or PEG is first carboxylated by reacting it with acetic anhydride in anhydrous solvent such as DMF.

A. Synthesis of 3-Nitrophenyl Activated Carrier Substance.

In a 100 mL round-bottom flask equipped with a magnetic stirrer and a nitrogen inlet is placed the carboxylated carrier substance such as pendant PEG with about 15 acid groups (1.00 g, 0.75 mmol acid) and 3-nitrophenol (0.14 g, 1.0 mmol). The mixture is dissolved in 10 mL of dry THF and cooled to 0° C. before a 10 mL THF solution of DIC (0.13 g, 1.0 mmol) and DMAP (0.012 g, 0.10 mmol) is added drop wise via a syringe over a 10 min period. The mixture is allowed to warm gradually to room temperature and stirred at this temperature for 18 h. The urea byproduct is filtered off and the filtrate is precipitated from isopropanol to recover the produce.

B. Synthesis of N-Succinimidyl Activated Carrier Substance.

In a 100 mL round-bottom flask equipped with a magnetic stirrer and a nitrogen inlet is placed the carboxylated carrier substance such as pendant PEG with about 15 acid groups (1.00 g, 0.75 mmol acid) and N-hydroxysuccinimide (0.12 g, 1.00 mmol). The mixture is dissolved in 5 mL of dry DMF and cooled to 0° C. before a 5 mL DMF solution of DIC (0.13 g, 1.0 mmol) and DMAP (0.012 g, 0.10 mmol) is added drop wise via a syringe over a 10 min period. The mixture is allowed to warm to room temperature and stirred for 18 h at this temperature. The urea byproduct is filtered off and the filtrate is precipitated from isopropanol to recover the produce.

C. Synthesis of S-Ethyl Activated Carrier Substance.

In a 100 mL round-bottom flask equipped with a magnetic stirrer and a nitrogen inlet is placed the carboxylated carrier substance such as pendant PEG with about 15 acid groups (1.00 g, 0.75 mmol acid) and ethanethiol (0.06 g, 1.00 mmol). The mixture is dissolved in 10 mL of dry THF and cooled to 0° C. before a 10 mL THF solution of DIC (0.13 g, 1.0 mmol) and DMAP (0.012 g, 0.10 mmol) is added drop wise via a syringe over a 10 min period. The mixture is stirred for 18 h at 0° C. The urea byproduct is filtered off and the filtrate is precipitated from isopropanol to recover the product.

D. Activated Ester Intercalator or Other Moiety.

With suitable modifications, the procedures used to add activated esters to the carboxylated carrier substances described previously, can also be used to add activated esters to carboxylated intercalators or other moieties. If needed, an intercalator or other moiety with a hydroxyl or amino group is first carboxylated by reacting it with acetic anhydride in anhydrous solvent such as DMF. These activated intercalators are then coupled to amino-derivatized carrier substances.

E. Coupling an Activated Intercalator to Amino-Containing Carrier Substances.

This procedure is used to conjugate amino-containing carrier substance (i.e. protein, amino-PEG or amino-dextran) with any suitable activated ester moiety including intercalators that have active ester (i.e. NHS) or isothiocyanate functional groups. At pH 9, conjugation occurs virtually exclusively at the amino group.

About 0.2 mmoles of amino-containing carrier substance (i.e., with about 0.1-0.2 mmoles of free primary amines) is dissolved in 1-2 mL of sterile distilled water. To this carrier solution is added 0.1-0.2 mL of 10×conjugation buffer (1M NaHCO₃/Na₂CO₃, pH 9).

A 10 mg/mL DMF solution is freshly prepared of the activated intercalator or moiety containing active ester or isothiocyanate. To the buffered carrier solution is added 0.2-0.4 mL of the DMF solution, mixed and allowed to stand at least 2 hours or overnight.

The reaction mixture is desalted on a column of Sephadex G-25 in water to remove the excess moiety. The product can be purified using reverse phase HPLC if necessary.

### Amination Methods

Carrier substances that do not normally contain amino groups can be suitably aminated to provide them by methods well known in the art as is disclosed for CD derivatives by A. R. Khan, et al, in Chem. Rev. 98, 1977-1996 (1998) and references therein which are hereby incorporated.

For instance, carrier substances such as carbohydrates including dextrans and cyclodextrins, as well as PEG and other hydroxlated polymers with available hydroxyl groups are readily aminated through tosylation. The hydroxyl groups are first reacted with p-toluene sulfonyl chloride, in suitable anhydrous solvent. Then the tosylate on the reactive site is displaced by treatment with excess sodium azide. Finally, the azide is reduced to an amine with an appropriate hydrogenation method such as with hydrogen and a noble metal catalyst to provide an amino-containing carrier substance.

### Thiolation Methods

On amino-containing carrier substances, intercalators and other moieties, the hydrazine or other amino groups can be thiolated to provide thiols for disulfide coupling such as between any suitable thiolated carrier substance and thiolated intercalator. Suitable methods using SPDP or 2-iminothiolane are disclosed by E. J. Wawrzynczak, et al, in C. W. Vogel (ed.) Immunoconjugates; Antibody Conjugates in Radioimaging and Therapy of Cancer. NY; Oxford Univ. Press, pp 28-55, 1987).

For instance, primary amino groups on the carrier substance are thiolated in PBS, pH 7.5 by adding a 2×molar excess of SPDP in EtOH and letting it react for about 1 hour at rt. Excess SPDP is removed by size exclusion gel chromatography. Before coupling, the pyridine-2-thione is released by adding a molar excess of DTT to provide sulfhydryl groups.

### Intercalation Methods

These are general methods for coupling nucleic acids to carrier substances with coupled intercalators to produce intercalator-linked coupling of the nucleic acid. Preferably, intercalation is done in a small volume of water at a salt concentration of less than 20 mM, preferably 1-10 mM salt, pH 6-8, at room temperature. Based on previous determinations of intercalator concentration that is coupled to the carrier substance, an excess molar concentration of nucleic acid vs. intercalator is combined with the carrier.

For instance, for each micromole of coupled psoralen available on the carrier in 20 microliters of 0.002 M NaCO₃, 1.5-3 micromoles of oligodeoxynucleotide is added in about 20 microliters of water. Intercalation was allowed to proceed for about 1-2 hours at rt in the dark.

With photoreactive intercalators such as psoralen or trioxsalen, the intercalator-linkages can be converted into covalent linkages. For instance, the intercalated mixture is irradiated with 365 nm uv light (8 watt lamp about 6 cm above the solution surface) for about 15-45 minutes at rt. If desired, the optimal irradiation time for a given mixture can be determined empirically by comparing preparations using gel migration inhibition as described below.

The nucleic acid-loaded carrier is purified by collecting the leading fractions during size exclusion gel chromatography on a column of Sephadex G-50 in water or MetOH in water (i.e. 30-50% MetOH). Alternatively, the product can be purified by suitable precipitation methods or by using reverse phase HPLC if necessary.

### Preparation I

### Psoralen Aldehyde Using Glycidol

To 12.5 micrograms (0.03 micromoles) of psoralen (Ps) amine (Sigma-Aldrich, St. Louis, Mo., Cat# P6100) was added 0.050 mL of N,N dimethyl formamide (DMF), then heated at 65° C. for 3 minutes to dissolve. To the psoralen amine solution was added about 0.013 mL of glycidol (Sigma-Aldrich, 960/%), vortexed and put in the dark at room temperature (rt) for about 48 hours to allow coupling of the glycidol to the amino groups. The psoralen-glycidol preparation was oxidized by adding 0.10 mL of about 0.16% NaIO₄ in water, mixed and left in the dark at rt for about 25 minutes to produce aldehyde groups.

The resulting psoralen-aldehyde preparation was purified on a 0.5 gm, C₁₈ solid phase extraction (SPE) column (Alltech Assoc., Deerfield, Ill.). The column was preconditioned with 3 mL of methanol (MetOH) and 3 mL of H₂O. The 0.13 μL psoralen-aldehyde preparation was added to the column and then washed with about 1 mL of water. The psoralen-aldehyde was eluted with about 1.2 mL of 100% MetOH and stored in the dark.

Alternatively, the amino group on the intercalator can be thiolated using 2-iminothiolane to provide thiolated psoralen for disulfide coupling to any suitable thiolated carrier substance.

### Preparation II

### Trioxsalen Aldehyde Using Solid Phase

An SPE column containing 500 mg of C₁₈ solid phase was preconditioned with 5 mL of MetOH, then 6 mL of water. Then about 0.125 mg (=0.000426 mmoles) of trioxsalen (Tx) amine (4′-aminomethyl trioxsalen, Calbiochem), in 0.1 mL of DMSO was applied and allowed to soak into the column bed, followed with about 5 mL of water.

Then 0.6 mL of 12.5% glutaraldehyde solution (for 1.5×) (previously adjusted to pH 10 with 1 M NaCO3) was applied and allowed to sit for about 40 minutes. The column was then washed with about 5 mL of water followed by 3 mL of 5% MetOH in water to remove uncoupled glutaraldehyde. The glutaraldehyde-coupled trioxsalen was then eluted with 2.5 mL of 100% MetOH and concentrated by evaporation in the dark.

The glutaraldehyde coupled trioxsalen was tested for purity using HPLC with an Xterra C₁₈ column (Waters Corp., Chicago Ill.) and a mobile phase of 15% acetonitrile in 25 mM ammonium formate, pH 6.5, flow rate 1 mL per minute. Purity was indicated by characteristic retention times when monitored by absorbance at 260 nm and refractive index.

Trioxsalen concentration was determined by fluorescence and aldehyde concentration was determined using HSD as described previously.

### Preparation III

### Psoralen Dextran Conjugates

In this example, fluorescein (FITC) labeled dextran is derivatized using glycidol and oxidation to provide aldehyde groups for coupling to psoralen amine and other moieties.

A. To 1 mL of 15% dextran, average mw 40,000 Daltons (40 kDa) (Sigma-Aldrich), was added 0.1 mL of 1 M NaCO3 to give a pH of about 12. To this solution was added about 0.012 mL of glycidol (40×molar), then put in the dark at rt for several days. The resulting dextran-glycidol preparation was oxidized by adding 0.05 gm of NaIO₄ and put in the dark at rt for about 2 hours. The resulting dextran-aldehyde was collected by precipitation with about 5 volumes of 100% isopropanol, cooling to −20° C. and centrifugation. The dextran-aldehyde precipitate was dissolved in water. Alternatively, it can be further purified by Sephadex™ G50 size exclusion gel chromatography in water. Aldehyde concentration is determined using HSD as described previously.

B. Psoralen amine was coupled to the dextran-aldehyde by adding about a two fold (2×) molar excess of psoralen amine to the dextran-aldehyde in water and put in the dark for several hours at rt. The resulting dextran-psoralen conjugate is purified by Sephadex™ G50 size exclusion gel chromatography in water.

C. Preparation of psoralen-dextran-poly arginine conjugate. Poly arginine (Sigma-Aldrich P4663, mw 10 kDa) was coupled to the remaining aldehydes on the dextran-aldehyde by adding about a two or three fold molar excess of poly arginine (i.e. 1.6 micromoles in 0.32 mL water), to about 0.8 micromoles of dextran-aldehyde in 0.5 mL water and about 0.040 mL of 0.02 M NaCO₃ for pH 8-9. The solution was mixed and put in the dark for several hours at rt. The resulting psoralen-dextran-poly arginine conjugate is purified by Sephadex™ G50 size exclusion gel chromatography in water or 50% MetOH in water.

Dextran concentration was measured as carbohydrate by a colorimetric test described previously. Poly arginine concentration was measured as amine by a colorimetric test for amines as described previously. Psoralen concentration is determined by fluorescence as described previously.

Nucleic acid loaded carrier is prepared by the intercalation method described previously, combining psoralen-dextran with suitable nucleic acid (i.e. ODN) at a molar ratio of 1:2 in water. The mixture is then uv irradiated before Sephadex™ G50 purification as disclosed previously.

### Preparation IV

### Psoralen Cyclodextrin Conjugates

In this example, a cyclodextrin (CD) preparation can be alpha, beta, or gamma cyclodextrin monomers, or dimers, trimers or polymers thereof. Also, a cyclodextrin preparation can be CD monomers, dimers, trimers or polymers previously coupled with glycidol.

To the glycidol treated CD preparation in water (4% CD), sodium m-periodate (NaIO₄) was added directly while mixing at room temperature (rt.). The molar ratio of NaIO₄ to cyclodextrin was about 6:1, to oxidize the diols introduced with the glycidol and some of the secondary C₂-C₃ diols on the CD molecules. This produces multiple aldehydes per CD molecule. The reaction is continued at 30° C. in the dark for 6 hours to overnight. The resulting polyaldehyde CD preparation was purified by gel exclusion chromatography (G50 Sephadex™) in water, and concentrated by evaporation.

A. Psoralen amine was coupled to the CD-aldehyde by adding about a two fold (2×) molar excess of psoralen amine to the CD-aldehyde in water and put in the dark for several hours at rt.

The resulting CD-psoralen conjugate is purified by Sephadex™ G50 size exclusion gel chromatography in water.

B. Poly arginine (Sigma-Aldrich P4663, mw 10 kDa) is coupled to the remaining aldehydes on the CD-aldehyde by adding about a two or three fold molar excess of poly arginine to the CD-aldehyde in water and put in the dark for several hours at rt. The resulting psoralen-CD-poly arginine conjugate is purified by Sephadex™ G50 size exclusion gel chromatography in water or 50% MetOH in water.

Cyclodextrin content is determined as carbohydrate as described previously. Poly arginine concentration is determined as amine as described previously. Psoralen concentration is determined fluorometrically as described previously.

Nucleic acid loaded carrier is prepared by the intercalation method described previously, combining psoralen-CD with suitable nucleic acid (i.e. ODN) at a molar ratio of 1:2 in water. The mixture is then uv irradiated before Sephadex™ G50 purification as disclosed previously.

### Preparation V

### Psoralen Lipid Conjugates And Micelles

In this example, psoralen amine is coupled to oleic acid by two different coupling methods. To each of two tubes (A and B), containing about 12.5 micrograms (0.03 micromoles) of psoralen amine (Sigma-Aldrich, Cat# P6100) was added 0.060 mL of DMF, then heated at 65° C. for 3 minutes to dissolve.

A. To psoralen amine solution A, was added about 0.023 mL of 1:5 CH₂Cl₂: DMF containing about 0.045 micromoles of oleic anhydride (Sigma-Aldrich), vortexed and put in the dark at rt for about 24 hours to allow coupling of the oleic anhydride to the amino groups.

B. To psoralen amine solution B, was added about 0.023 mL of DMF containing about 0.045 micromoles of oleic acid N-hydroxysuccinimide ester (Sigma-Aldrich), vortexed and put in the dark at rt for about 24 hours to allow coupling of the oleic acid N-hydroxysuccinimide ester to the amino groups.

Both preparations A and B were quenched with about 0.003 mL of ethanolamine (0.05 micromoles), vortexed and put in the dark at rt for about 24 hours. The resulting psoralen-oleic acid conjugates were purified by chromatography on C₁₈ columns using gradient elution of 10-100% acetonitrile in water. Psoralen concentration is determined by fluorescence with 340 nm excitation wavelength and emission at 528 nm using least squares calculation from a psoralen standard curve. Preparations were stored at −20° C.

C. Nucleic acid loaded carrier is prepared by the intercalation method described previously, combining psoralen-lipid with suitable nucleic acid (i.e. ODN) at a molar ratio of 1:2 in water. The mixture is then uv irradiated before Sephadex™ G50 purification as disclosed previously. This preparation is incorporated into any suitable micelle or liposome formulation which can include other amphiphilic molecules as disclosed herein to provide the micelle or liposome carrier composition of this invention.

### Preparation VI

### Psoralen Antibody Conjugate

In this example (Nat23), psoralen is coupled to antibody protein to provide a psoralen-protein carrier. Nucleic acid is then coupled to the antibody through intercalation linkages between the psoralen and nucleic acid.

A. Antibody Coupling.

To about 1 mg of goat anti-human IgG antibody (Sigma-Aldrich 13382) in 1.6 mL of 0.002 M NaCO₃, pH 8-9, was added about 0.56 micromoles of succinimidyl-[4-(psoralen-8-yloxy)] butyrate (SPB, Pierce Cat #23013), in 0.012 mL of DMF. The mixture was vortexed and put in the dark at rt for about 24 hours to couple the SPB to the amino groups on the antibody.

B. Purification.

The resulting psoralen-antibody conjugate is purified by Sephadex™ G50 size exclusion gel chromatography in water. Psoralen concentration is determined by fluorescence as described previously.

Alternatively, psoralen-aldehyde or trioxsalen-aldehyde can be coupled to the antibody through available amino groups on the protein.

In another embodiment, the carbohydrate moiety of antibody or antibody fraction, is suitably oxidized to aldehyde using either NaIO4 (A. Murayama, et al, Immunochem. 15, 532, 1978), or a suitable oxidizing enzyme. Then, psoralen amine or trioxsalen amine is coupled to the aldehydes on the antibody.

In another embodiment, the sulfhydryl moiety of an antibody fraction is suitably coupled to the intercalator. For instance, thiolated psoralen or thiolated trioxsalen is coupled to the sulfhydryl on the antibody using a dithiol linkage.

C. Intercalation.

The resulting psoralen-antibody conjugate was coupled to DNA by intercalator-linked coupling. To about 12 micrograms of psoralen-antibody conjugate in 20 microliters of 0.002 M NaCO3 was added an aliquot of 2 micrograms of Lambda “marker” DNA fragments (EcoR1+Hind III digest, average 3731 base pair fragments, Promega Cat #G173A) in 16 microliters of water. Intercalation was allowed to proceed for about 1.5 hours at rt in the dark. This preparation was divided into two aliquots. Aliquot A, as the control, was kept in the dark at rt. The test sample, aliquot B was then irradiated with 365 nm uv light (8 watt lamp about 6 cm above the surface) for 25 minutes at rt to produce covalent linkages.

D. Gel Migration Inhibition Assay.

A 0.6% agarose gel (NuSieve 3:1 agarose, FMC Bioproducts, Rockland, Me.) was prepared horizontally in 89 mM Tris borate, 2 mM EDTA buffer, pH 8.3 (TBE, Sigma-Aldrich Cat #T9525), containing 1 microgram per mL of ethidium bromide. To 0.036 mL each of samples A and B was added 0.010 mL of 10× gel-loading solution (20% Ficoll®, 1% SDS, 0.2% bromophenol blue in water) and 0.030 mL of each mixture was loaded into wells in the agarose gel. Agarose gel electrophoresis (AGE) was run for about 2 hours at 60 volts. DNA bands in the gel were visualized by fluorescence over a uv transilluminator. The gel was photographed and band migration distances were measured.

The results showed control sample A had a thin zone of DNA in the well with about four bands of DNA that migrated 1.4, 2.5, 2.7 and 2.9 cm from the well. The uv treated, psoralen-antibody sample B had a very heavy zone of DNA in the well with only two faint bands at 2.6 and 2.7 cm from the well. The results indicated that most of the DNA in sample B remained in the well and could not migrate to form bands as was seen in the control. This is evidence that the DNA in sample B was coupled to the psoralen-antibody conjugate.

### Preparation VII

### Psoralen Peptide Conjugate

In this example (Nat25), psoralen is coupled to a polythreonine peptide to provide a psoralen-peptide carrier. Nucleic acid is then coupled to the peptide through intercalation linkages between the psoralen and nucleic acid.

A. Coupling.

To about 25 mg of poly-L-threonine (Sigma-Aldrich P8077) in 3.5 mL of about 0.001 M NaCO3 in 3.5 mL of 60% DMF, pH 8-9, was added about 16.5 micromoles of succinimidyl-[4-(psoralen-8-yloxy)] butyrate (SPB, Pierce Cat #23013), in 0.33 mL of DMF. The mixture was vortexed and put in the dark at rt for about 24 hours to allow coupling of the SPB to the amino groups on the poly threonine.

B. Purification.

The resulting psoralen-peptide conjugate is purified by Sephadex™ G50 gel exclusion chromatography in water. Psoralen concentration is determined by fluorescence as described previously.

Alternatively, psoralen-aldehyde or trioxsalen-aldehyde can be coupled to the peptide through available amino groups on the peptide. Also, any suitable peptide with one or more available amino groups as disclosed herein, can be substituted for the polythreonine in this example.

C. Without vs. With Intercalation of DNA.

The resulting psoralen-peptide conjugate was mixed with DNA under two conditions. In sample A, high salt concentration (i.e. >20 mM salt) was used to suppress intercalation. In sample B, low salt concentration allows intercalation and intercalator-linked coupling.

A non-intercalated control A solution was prepared with an aliquot of about 10 micrograms of psoralen-peptide conjugate in 15 microliters of 1 mM NaCO₃ to which was added an aliquot of 1.5 micrograms of Lambda “marker” DNA fragments, (Promega Cat #G173A) in 12 microliters of water. To suppress intercalation in control A with salt, 3 microliters of 1 M NaCl was added (total volume 20 microliters).

A sample B solution was prepared for intercalation with a second aliquot of about 10 micrograms of psoralen-peptide conjugate in 15 microliters of 1 mM NaCO₃ to which was added an aliquot of 1.5 micrograms of Lambda “marker” DNA fragments, (Promega Cat #G173A) in 12 microliters of water, plus 3 more microliters of water (total volume 20 microliters).

Both preparations were left for about 2 hours at rt in the dark. Aliquot A, as the control, was kept in the dark at rt. Sample B was irradiated with 365 nm uv light (8 watt lamp about 6 cm above the surface) for 15 minutes at rt to produce covalent linkages.

D. Gel Migration Inhibition Assay.

A 0.6% agarose gel was prepared as before, containing 1 microgram per mL of ethidium bromide. To 0.030 mL each of samples A and B was added 0.010 mL of 10× gel-loading solution and 0.030 mL of each mixture was loaded into wells in the agarose gel as before. AGE was run for about 1 hour at 60 volts. DNA bands in the gel were visualized by fluorescence over a uv transilluminator. The gel was photographed and band migration distances were measured.

The results showed control sample A had a thin zone of DNA in the well with about five strong bands of DNA that migrated 0.4, 0.7, 1.0, 1.1 and 1.3 cm from the well. The uv treated, psoralen-peptide sample B had a very heavy zone of DNA in the well with only one band immediately below the well. Most of the DNA in sample B remained in the well and could not migrate to form bands as was seen in the control. This is evidence that the DNA in sample B was coupled to the psoralen-peptide conjugate.

### Preparation VIII

### Psoralen PEG Conjugate

In this example (Nat26), psoralen amine is coupled to a diepoxy PEG to provide a psoralen-PEG carrier. The carrier is then thiolated to provide sulfhydryl groups for coupling other moieties. Nucleic acid can then be coupled to the PEG through intercalation linkages between the psoralen and nucleic acid.

B. Coupling.

To about 12.5 micrograms (0.03 micromoles) of psoralen amine (Sigma-Aldrich P6100) in 0.20 mL of DMF was added about 700 micrograms (0.03 micromoles) of polyethylene glycol diglycidyl ether, “PEG-DE”, mw about 23,250 (Sigma-Aldrich #47,569-6). The solution was mixed and put in the dark at rt for about 3 weeks.

Remaining epoxy groups were quenched by adding 30 micrograms (0.12 micromoles) of sodium thiosulfate in 0.010 mL water, mixed and kept at rt in the dark for 2 days. To this solution was added about 0.23 milligrams of dithiothreitol (DTT) in about 1 mL of water, mixed and kept at rt in the dark for about 2 hours to reduce coupled sodium thiosulfate to sulfhydryl groups on the psoralen-PEG conjugate.

B. Purification.

The preparation was fractionated by size exclusion gel chromatography on a 2.5 cm diameter×18.3 cm long column of Sephadex™ G25 with 0.005 M HCl, pH 2.5 as the mobile phase. Fractions were collected and monitored for psoralen fluorescence as described previously. The leading fractions contained PEG with psoralen fluorescence, indicating that psoralen was coupled to the PEG. The psoralen-PEG fractions were pooled and concentrated by evaporation in the dark, under flowing nitrogen.

Alternatively, the PEG-DE is first coupled to hydrazine through the epoxy groups to produce amino-PEG. Then aldehyde derivatized psoralen or aldehyde derivatized trioxsalen is coupled to the hydrazine on the PEG to provide acid labile linkages as described previously. Alternatively, any suitable diamino compound can be used in place of hydrazine, and/or psoralen-amine or trioxsalen-amine can be coupled to the amino-PEG through suitable cross linkers.

Alternatively, the PEG-DE is first coupled to sodium thiosulfate through the epoxy groups, then reduced with DTT to produce sulfhydryl-PEG. Then sulfhydryl derivatized (thiolated) psoralen or sulflhydryl derivatized (thiolated) trioxsalen is coupled to the sulfhydryl groups on the PEG to provide dithiol biocleavable linkages as described previously.

C. Intercalation.

In any case, nucleic acid is coupled to the PEG carrier through intercalation linkages between the psoralen (or trioxsalen) and nucleic acid as disclosed previously.

### Preparation IX

### Pendant PEG with Hydrazine Functional Groups

In this example, pendant polyethylene glycol (SunBio USA, mw 20 KDa) with approximately 15 propionic acid side chains (PaPEG)is coupled to hydrazine through available carbonyl groups on the PEG. This provides side chains with terminal hydrazine moieties. The hydrazine groups can then be coupled to aldehyde groups to provide acid-labile hydrazone linkages.

A. Coupling.

Into about 20 ml of water, about 5 gm of pendant PEG was dissolved, the pH was about 5.

Based on the manufacturer's value of 15 moles of propionic acid per mole of PaPEG, there was about 0.375 mmoles of carboxylic acid present. In a separate container, 1.8 ml of hydrazine hydrate (64%, fw 50.06) was neutralized to pH 7 with about 6.25 ml of 5N HCl, to give a final concentration of about 0.225 ml hydrazine per ml of solution.

A thirty-fold molar excess (30×) of hydrazine (4 ml of hydrazine solution) was added to the PaPEG solution and mixed with a magnetic stirrer. After about 2 minutes, a twenty-fold molar excess (20×=1.45 gm) of N-(3-Dimethylaminopropyl)-N′-Ethylcarbodiimide (EDC, fw 191.7), was added to the solution of PaPEG and mixed thoroughly. The pH was about 6. The solution was allowed to react overnight at room temperature (rt).

B. Purification.

The reaction mixture was fractionated on a Sephadex™ G25 column equilibrated and eluted with 0.005 M HCl in water. The fractions were analyzed for refractive index. They were also analyzed for primary amine using a colorimetric test described previously. The leading fractions with corresponding high refractive index and amine content were pooled and concentrated by evaporation under nitrogen gas. The resulting product (PaPEG-Hzn), is PaPEG with hydrazine functional groups covalently coupled to the propionic acid moieties.

The PaPEG-Hzn can now have any suitable intercalator with a terminal aldehyde group coupled to the available hydrazine groups. This will provide an acid labile hydrazone linkage described herein. Alternatively, any suitable diamino compound can be used in place of hydrazine.

Alternatively, any suitable intercalator with a terminal active ester can be coupled to the amine as described herein. Also, using suitable bifunctional amino coupling agents described herein, any suitable amino derivatized intercalators can be covalently coupled to the hydrazine (or amino) moieties.

Alternatively, the hydrazine (or amino) groups can be thiolated using SPDP or 2-iminothiolane as described herein to provide thiols for disulfide coupling to any suitable thiolated intercalator.

Also, using coupling agents described herein, the terminal hydrazine groups can be coupled to a diamino, Fmoc half-protected biocleavable peptide containing any suitable biocleavable sequence such as GFLG, Phe-Leu, Leu-Phe or Phe-Phe, among others. The Fmoc groups are then removed to provide unprotected amino groups for subsequent coupling to an intercalator.

Alternatively, said biocleavable peptide can include a sulfhydryl group at one end for subsequent coupling to a thiolated intercalator (i.e. disulfide coupling), or amino-derivatized intercalator using a bifunctional cross linking agent.

Alternatively, the hydroxyl end groups on the PEG backbone can be suitably derivatized and coupled to suitable targeting molecules, transduction vectors, or grafted polymers using other coupling groups such as succinimide, N-succinimidyl, bromoacetyl, maleimide, N-maleimidyl, oxirane, p-nitrophenyl ester, or imidoester. Also, aldehydes that are coupled to hydrazine to give amino-aldehyde (Schiff's base) bonds can be reduced with NaBH₄ to stabilize them.

### Preparation X

### Coupling PaPEG-Hzn to Aldehyde-Trioxsalen

In this example, trioxsalen (Tx) with terminal aldehyde groups is coupled to the available hydrazine groups on the PEG to provide acid labile hydrazone linkages.

A. Coupling.

The glutaraldehyde-coupled trioxsalen is combined with a slight molar excess of PaPEG-Hzn based on amino content (0.0228 mmoles amino in 0.2 mL added) vs. Tx aldehyde concentration. The reaction mixture is allowed to proceed for about 2 hours and concentrated by evaporation in the dark, under flowing nitrogen.

The PEG-trioxsalen (PEGTx) is purified by precipitation with 100% isopropanol at −20° C. and centrifugation. The pellet is dissolved in 2 mL of water with 2 minute sonication and fractionate on a Sephadex™ G25 “mini” column (bed=0.7×4.8 cm) with 50% MetOH in water (or 2 mM NH₄ formate, pH 7).

The fractions are monitored for psoralen fluorescence with 340 nm excitation wavelength and emission at 528 nm. The leading fractions with the highest fluorescence are pooled and concentrated by evaporation in the dark, under flowing nitrogen.

B. Intercalating Pendant PEG-Tx to ODN.

The PEG-trioxsalen is first allowed to intercalate with oligodeoxynucleotide (ODN) and then UV irradiated to provide covalent linkages. The intercalation is preferably done in pure water or water diluted to less than 20 mM salt concentration.

The nucleic acid used in this example was a commercially prepared, phosphorothioate anti-bc12 antisense ODN (G3139), that had a 5′ extension of phosphodiester thymidines with an 5′ FITC label and terminal amino group. The sequence and composition are as follows;
Phosphodiester Extension|Phosphorothioate G3139 antisense bcl2

5′-Amino-Flour-TTT TTT TCT TTT TTT TCT CCC AGC GTG CGC CAT-3′

In a microfuge tube, the ODN was combined with a slight molar excess of PaPEG-Tx based on trioxsalen concentration. The intercalation is allowed to proceed for about 1 hour in the dark. The mixture can then be exposed to UV irradiation to form covalent bonds. This was done by putting the open tube under a UV lamp (365 nm, 8 watt) about 2-3 cm below the lamp for 15 minutes.

The PEG-ODN conjugate is purified by precipitation with 100% isopropanol at −20° C. and centrifugation. The pellet is dissolved and fractionated on a Sephadex™ G25 “mini” column (bed=0.7×4.8 cm) with 50% MetOH in water.

The fractions are monitored for fluorescein fluorescence with 485 nm excitation wavelength and emission at 528 nm. The leading fractions with the highest fluorescence are pooled and concentrated by evaporation in the dark, under flowing nitrogen. The PEG-ODN conjugate is characterized for purity and for molecular weight using HPLC as described previously.

### Preparation XI

### Coupling Through Thiol Groups to Pendant PEG

In this example, hydrazine-linked or diamino-linked PaPEG described previously is thiolated before coupling through disulfide linkages to a thiolated nucleic acid intercalator, targeting molecule, transduction vector, or other moiety.

The amino groups on the PaPEG are thiolated in PBS, pH 7.5 by adding a 2×molar excess of SPDP in EtOH and letting it react for about 1 hour at rt. Excess SPDP is removed by size exclusion gel chromatography. Before coupling, the pyridine-2-thione is released by adding a molar excess of DTT to provide sulfhydryl groups. Alternatively, other suitable amino-containing carrier substances can be substituted for the PaPEG.

### Preparation XII

### Maleimido or Iodo Carrier Substances Coupled to a Thiolated Moiety

In this example, an amino-containing carrier substance is derivatized to contain a maleimide or an iodo reactive group. Then an intercalator, targeting molecule, transduction vector or other moiety is suitably thiolated as described herein before coupling it to the derivatized carrier substance. There are well known methods for derivatizing the primary amine on the carrier substance (i.e. protein, PEG) to provide a maleimido group. For instance, a bifunctional (succinimidyl-maleimido) cross linker described herein, such as MBS or SMPB is coupled to the primary amine to provide free maleimide groups. Upon reaction with a thiolated moiety, a stable thioether bond is formed.

Alternatively, iodo-carrier substances such as iodo-polyethylene glycol (Iodo-PEG) carriers can be prepared for coupling to a sulfhydryl group on an intercalator, targeting molecule, transduction vector or other moiety. For instance, NHS esters of iodoacids can be coupled to the amino-containing carrier substances. Suitable iodoacids for use in this invention are iodopropionic acid, iodobutyric acid, iodohexanoic acid, iodohippuric acid, 3-iodotyrosine, among others. Before coupling to the amino-carrier substance, the appropriate Iodo-NHS ester is prepared by known methods. For instance, equimolar amounts of iodopropionic acid and N-hydroxysuccinimide are mixed, with suitable carbodiimide, in anhydrous dioxane at RT for 1-2 Hrs, the precipitate removed by filtration, and the NHS iodopropionic acid ester is collected in the filtrate. The NHS iodopropionic acid ester is then coupled to the amino-carrier substance.

### Preparation XIII

### Amphiphilic Cyclodextrin

In this example, a mixture of amphiphilic cyclodextrin dimers, trimers and polymers with alkyl carbon chains attached is prepared for use as carrier substances. The cyclodextrins are cross-linked through hydroxyl groups using 1,4 butanediol diglycidyl ether (BDDE). Excess BDE molecules coupled at one end to the CD provide terminal oxirane groups that are subsequently thiolated by reaction with thiosulfate and reduction. Alkyl carbon chains are coupled to the CD derivatives using a “long chain epoxy” that couples to other available hydroxyl groups (CD88).

A. Cross-Linking with BDDE.

Into 125 ml of hot water (70-80° C.) adjusted to pH 4.5-5 with 0.05 ml 6 N HCl, is dissolved 2.84 gm of beta cyclodextrin (0.0025 moles). To this solution 4.1 ml of BDDE (about 0.0125 moles) is added with mixing and continued heating for about 2 hours.

B. Coupling with a Long Chain Epoxy.

The mixture is adjusted to pH>10 with 1 M KOH and 1.28 gm (about 0.005 moles) of dodecyl/tetradecyl glycidyl ether (DTGE) is added and mixed vigorously. The solution is periodically mixed for about 1.5 hours, heated for about 3 hours and then left at room temperature (rt) overnight. The resulting solution is light yellow and turbid.

C. Thiolation with Na Thiosulfate.

To the reheated mixture, 6 gm (about 0.025 moles) of sodium thiosulfate is added and mixed. After about 1 hour, the pH is adjusted to 7 with KOH and the solution was heated for about 3.5 hours more. Excess DTGE was removed by chilling to solidify the DTGE and the solution was decanted. The mixture was dialyzed against a continuous flow of distilled water in 500 molecular weight cutoff (mwco) tubing (Spectra/Por CE) for about 40 hours. The solution was concentrated by evaporation to 8 ml to give a clear, light yellow solution.

To the mixture, 8 ml of water and 0.96 gm (about 0.0062 moles) of dithiothreitol (DT) was added, mixed and left overnight. The turbid solution was then dialyzed against a continuous flow of distilled water in 500 mwco tubing (Spectra/Por CE) for about 40 hours. The solution was concentrated by evaporation to 3.7 ml to give a clear, yellow solution. Total yield based on dry weight was 2.276 gm.

D. Column chromatography and testing.

The mixture was fractionated on a Sephadex™ G15 column (2.5×47 cm) in water. The fractions were tested for relative carbohydrate and thiol concentration as described previously.

Fractions with corresponding peak concentrations of carbohydrate and thiol were pooled and concentrated by evaporation. The final volume was 2.2 ml and the total yield based on dry weight was 1.144 gm. The resulting amphiphilic CD polymer was highly water soluble and amorphous (glassy) when dried.

E. Coupling With Thiolated Trioxsalen.

The amino groups on intercalators such as psoralen amine or trioxsalen amine and other moieties can be thiolated using SPDP or 2-iminothiolane as described previously. The thiolated intercalators are then coupled to the carrier substance through disulfide linkages using thiol-disulfide interchange as described previously. Also, other thiolated moieties such as targeting molecules, transduction vectors and grafted polymers can be coupled through disulfide linkages.

Alternatively, to produce other suitable hydrophobic CD derivatives, other alkyl chains can be introduced by substituting suitable alkyl epoxy compounds for the one used in this example. For instance 1,2-epoxy derivatives of any suitable alkane such as propane, butane, pentane, hexane, octane, decane and dodecane can be substituted. Other useful epoxies such as glycidyl isopropyl ether, glycidyl methacrylate and glycidyl tosylate can be substituted. Also certain aromatic epoxies or heterocyclic epoxies can be substituted such as benzyl glycidyl ether, (2,3-epoxypropyl) benzene, 1,2-epoxy-3-phenoxypropane, exo-2,3-epoxynorborane, among others.

Alternatively, the CD polymer can be suitably derivatized with other coupling groups such as succinimide, N-succinimidyl, bromoacetyl, maleimide, N-maleimidyl, oxirane, p-nitrophenyl ester, or imidoester. Also, the CD polymer can be coupled to a polypeptide containing any suitable biocleavable sequence such as Phe-Leu, Leu-Phe or Phe-Phe, among others. Also, the CD polymer can be suitably derivatized to provide a CD-block with an N carboxyanhydride for subsequent copolymerization into PEO-block copolymers.

Combinations for this invention can include the covalent coupling of an alpha CD with a beta CD, an alpha CD with a gamma CD, a beta CD with a gamma CD and polymers with various ratios of alpha, beta and gamma cyclodextrins.

F. Intercalation.

Nucleic acid loaded carrier is prepared by the intercalation method described previously, combining trioxsalen-CD with suitable nucleic acid (i.e. ODN) at a molar ratio of 1:2 in water. The mixture is then uv irradiated before Sephadex™ G50 purification as disclosed previously. This preparation is incorporated into any suitable micelle or liposome formulation which can include other amphiphilic molecules as disclosed herein to provide the micelle or liposome carrier composition of this invention.

### Preparation XIV

### Nucleic Acid Carriers From Hydroxylated Polymers

These are methods for synthesizing nucleic acid carrier compositions to provide for coupling to any suitable intercalator, targeting molecule, transduction vector, or other moiety with a suitable functional group. The targeting molecule can be a suitable protein, including antibodies, lectins, avidins and streptavidin, or ligands.

A. Preparation of NHS-Carrier Substances.

A carrier substance with terminal hydroxyl groups such as carbohydrates, PEG and other grafted polymers described herein, is derivatized to provide an NHS ester. In a suitable anhydrous solvent such as DMF, the carrier substance is coupled to acetic anhydride and purified as described herein, to provide carboxyl groups. Then, the carboxylic acid group is reacted with N-hydroxysuccinimide and an aromatic carbodiimide such as N,N-dicyclohexylcarbodiimide, at approximately equimolar ratios and reacted at rt for 1-3 Hrs. The product, N-hydroxysuccinimide carrier (i.e. NHS-PEG), is separated in the filtrate from precipitated dicyclohexylurea, collected by evaporation and purified by chromatography.

Under appropriate conditions, NHS-carrier substances can be prepared by coupling NHS esters directly to amino derivatized carrier substance. Preferably, the NHS ester is a bifunctional NHS coupling agent with a suitable spacer. Suitable NHS coupling agents for use in this invention have been previously described, including DSS, bis(sulfosuccinimidyl)suberate (BS3), DSP, DTSSP, SPDP, BSOCOES, DSAH, DST, and EGS, among others.

In any case, the NHS-carrier substance can now be coupled to any suitable amino-containing intercalator, targeting molecule, transduction vector, or other amino-containing moiety using methods for coupling active esters described herein.

B. Thiolated Carrier Substances.

Alternatively, thiolated carrier substances can be prepared from amino-containing carrier substances as described herein. Then, through disulfide coupling, the carrier substance is coupled to other available sulfhydryls on the desired thiolated intercalator, targeting molecule, transduction vector, or other moiety.

Alternatively, a sulfhydryl-containing carrier substance (i.e. thiolated PEG) is coupled to any maleimide derivative of an intercalator, transduction vector, targeting molecule, or biotin, (e.g. biotin-maleimide) or iodoacetyl derivatives such as N-iodoacetyl-N′-biotinylhexylenediamine.

C. Maleimido or Iodo-Carrier Substances.

Alternatively, maleimide or iodo derivatized carrier substances, can be prepared from amino-containing carrier substances of this invention using well known methods. Such carrier substances are suitable for coupling to native or introduced sulfhydryls on the desired intercalator, targeting molecule, transduction vector, or other moiety.

A maleimido group is added to an amino-carrier substance suitably prepared as described previously, by coupling a suitable hetero-bifunctional coupling agent to the available amino group. The hetero-bifunctional coupling agent consists of a suitable spacer with a maleimide group at one end and an NHS ester at the other end. Examples are previously described and include MBS, SMCC, SMPB, among others. The reaction is carried out so that the NHS ester couples to the available amino group on the carrier substance, leaving the maleimide group free for subsequent coupling to an available sulfhydryl on an intercalator, transduction vector, targeting molecule, or other moiety.

Under appropriate conditions, iodo-carrier substances (i.e. Iodo-PEG) can also be prepared for coupling to sulfhydryl groups. For instance, NHS esters of iodoacids can be coupled to the amino-carrier substances as described previously.

### Preparation XV

### Biotinylated Nucleic Acid Carriers

Carrier substances defined herein can be coupled to biotin by a variety of known biotinylation methods suitably modified for use with the carrier substances of this invention. For instance, an amino-containing carrier substance is combined with an active ester derivative of biotin in appropriate buffer such as 0.1 M phosphate, pH 8.0, reacting for up to 1 hour at room temperature. Examples of biotin derivatives that can be used are, biotin-N-hydroxysuccinimide, biotinamidocaproate N-hydroxysuccinimide ester or sulfosuccinimidyl 2-(biotinamino)ethyl-1,3′-dithiopropionate, among others.

Through the use of suitable protection and deprotection schemes, as needed, any carrier substance of the instant invention can be coupled to biotin or a suitable derivative thereof, through any suitable coupling group. For instance, biocytin can be coupled through an available amino group to any active ester derivatized carrier substance described herein.

The resulting biotinylated carrier substance is then coupled to any suitable avidin or streptavidin that contains the desired intercalator. The avidin or streptavidin may also contain a targeting molecule, transduction vector, or other moiety. In any case, the desired nucleic acid is coupled to the intercalator on the avidin or streptavidin using intercalation methods described herein.

### Preparation XVI

### Avidin Nucleic Acid Carriers

Avidin or streptavidin carrier substances defined herein can be coupled to biotinylated moieties including biotinylated intercalators. For instance, streptavidin can be suitably carboxylated without impairing the biotin binding sites. The carboxyl groups are then derivatized to provide one or more active esters as described herein.

Psoralen amine or trioxsalen amine is then coupled to the activated esters as described herein. Biotinylated moieties can also be coupled to the streptavidin carrier substance before or after nucleic acids are intercalated with the psoralen or trioxsalen. Biotinylated moieties can include targeting molecules or transduction vectors.

Alternatively, moieties such as targeting molecules or transduction vectors can be coupled to the active esters through their amino groups. Then, biotinylated intercalators such as psoralen or trioxsalen can be coupled to the biotin binding sites. In any case, the desired nucleic acid is coupled to the intercalator on the avidin or streptavidin using intercalation methods described herein.

### PREPARATION XVII

### Chloroquine Coupled Nucleic Acid Carrier

The prior art has shown that chloroquine given as free drug in high enough concentration, enhances the release of various agents from cellular endosomes into the cytoplasm. The purpose of this composition is to provide the chloroquine at the same site where the carrier needs to be released, thereby reducing the overall dosage needed.

A nucleic acid carrier composition has been discovered that includes the coupling of a chloroquine substance as defined herein, to any suitable active agent carrier composition including the nucleic acid carrier compositions of this invention.

Chloroquine Substance.

Chloroquine substance is defined herein to include chloroquine (7-chloro-4-(4-diethylamino-1-methylbutylamino) quinoline), chloroquine phosphate, chloroquine sulfate or a suitable quinoline or chloroquine derivative that enhances the release of various agents from endosomes into the cytoplasm. Chloroquine substance also includes hydroxychloroquine or any suitable hydroxy derivative of chloroquine such as disclosed by A. Surrey, et al, in JACS 72, 1814-1815 (1950), and references therein.

In one embodiment the coupling can be through noncovalent binding (i.e. entrapped within a CD, liposome or micelle carrier). In another embodiment the coupling is by covalent coupling. The chloroquine can also be coupled covalently to the carrier through a biocleavable linkage as defined herein.

In any case, the coupling is such that the chloroquine or chloroquine derivative is capable of promoting release of said carrier with an active agent, or an active agent alone, from within a cellular endosome or lysosome.

For example, hydroxychloroquine or any suitable hydroxy derivative of chloroquine, is coupled to a carrier substance defined herein (i.e. PEG, or antibody) through an ester bond using the available hydroxyl group coupled to a carboxylate on the carrier substance. Said carrier substance would also be coupled to any suitable active agent including a nucleic acid defined herein.

In another embodiment, a hydroxychloroquine derivative can be provided with a keto group through oxidation of said hydroxyl group directly. Or, hydroxychloroquine can be derivatized to provide an aldehyde by coupling glycidol to the hydroxyl group and then oxidizing the resulting vicinal hydroxyl groups with sodium periodate. The resulting aldehyde-chloroquine can then be coupled to any suitable amino group on the carrier substance. For instance, the aldehyde-chloroquine derivative can be coupled to hydrazine groups provided on the carrier substance to produce an acid-labile biocleavable linkage.

In another embodiment, a chloroquine derivative can include an amino group in place of, or coupled to, the hydroxyl group of hydroxychloroquine. For instance, hydroxychloroquine is first coupled with N-(2,3-epoxypropyl) phthalimide (EPP) to provide a protected amino coupled to the hydroxyl group. The phthalimide protective group is then removed by hydrolysis to provide a primary amine for subsequent coupling. Preferably, a suitable chloroquine derivative is coupled to the carrier substance through a biocleavable linkage as defined herein.

In another embodiment, a chloroquine derivative is suitably coupled directly to any suitable nucleic acid as described herein.

While the invention has been described with reference to certain specific embodiments, it is understood that changes may be made by one skilled in the art that would not thereby depart from the spirit and scope of the invention, which is limited only by the claims appended hereto.

1. A pharmaceutical nucleic acid carrier composition comprising;
a) a carrier substance covalently coupled to;
b) a nucleic acid intercalator and;
c) wherein said nucleic acid intercalator is coupled to a nucleic acid.

2. The composition of claim 1 wherein said nucleic acid intercalator of (b) is selected from the group consisting of photoreactive intercalators.

3. The composition of claim 1 wherein said nucleic acid is selected from the group consisting of single stranded RNA, double stranded RNA, antisense RNA, messenger RNA, transfer RNA, small interfering RNA, micro RNA, ribozymes, riboswitches, 5′ derivatized RNA, 3′ derivatized RNA, backbone derivatized RNA, single stranded DNA, double stranded DNA, 5′ derivatized DNA, 3′ derivatized DNA, oligonucleotides, phosphodiester sense and antisense oligonucleotides, phosphodiester sense and antisense oligodeoxynucleotides, backbone derivatized sense and antisense oligonucleotides, backbone derivatized sense and antisense oligodeoxynucleotides, mixed backbone derivatized sense and antisense oligonucleotides, mixed backbone derivatized sense and antisense oligodeoxynucleotides, RNA-DNA hybrids, modified ribose nucleic acids, locked nucleic acids, triplex-forming oligonucleotides, RNA-DNA chimeras, sense and antisense peptide nucleic acids, PNA clamps and phosphoric acid ester nucleic acids.

4. The composition of claim 1 further comprising a targeting molecule coupled to said carrier substance.

5. The composition of claim 1 further comprising a transduction vector coupled to said carrier substance.

6. The composition of claim 1 wherein said covalent coupling of said carrier substance of (a) to intercalator of (b) is a biocleavable linkage selected from the group consisting of a hydrazone linkage, a disulfide linkage, a protected disulfide linkage, an ester linkage, an ortho ester linkage, a phosphonamide linkage, a biocleavable polypeptide, an aromatic azo linkage and an aldehyde bond.

7. The composition of claim 1 further comprising a chloroquine substance coupled to said carrier substance.

8. A pharmaceutical nucleic acid carrier composition comprising;
a) a carrier substance noncovalently coupled to;
b) a nucleic acid intercalator and;
c) wherein said nucleic acid intercalator is coupled to a nucleic acid.

9. The composition of claim 8 wherein said nucleic acid intercalator of (b) is selected from the group consisting of photoreactive intercalators.

10. The composition of claim 8 wherein said nucleic acid is selected from the group consisting of single stranded RNA, double stranded RNA, antisense RNA, messenger RNA, transfer RNA, small interfering RNA, micro RNA, ribozymes, riboswitches, 5′ derivatized RNA, 3′ derivatized RNA, backbone derivatized RNA, single stranded DNA, double stranded DNA, 5′ derivatized DNA, 3′ derivatized DNA, oligonucleotides, phosphodiester sense and antisense oligonucleotides, phosphodiester sense and antisense oligodeoxynucleotides, backbone derivatized sense and antisense oligonucleotides, backbone derivatized sense and antisense oligodeoxynucleotides, mixed backbone derivatized sense and antisense oligonucleotides, mixed backbone derivatized sense and antisense oligodeoxynucleotides, RNA-DNA hybrids, modified ribose nucleic acids, locked nucleic acids, triplex-forming oligonucleotides, RNA-DNA chimeras, sense and antisense peptide nucleic acids, PNA clamps and phosphoric acid ester nucleic acids.

11. The composition of claim 8 wherein said carrier substance is selected from the group consisting of avidins, streptavidins, liposomes, micelles and dendrimers.

12. The composition of claim 8 further comprising a targeting molecule coupled to said carrier substance.

13. The composition of claim 8 further comprising a targeting molecule coupled to said carrier substance.

14. A method for synthesizing a pharmaceutical nucleic acid carrier composition comprising the steps of coupling;
a) a carrier substance to;
b) a nucleic acid intercalator to produce a carrier substance with coupled intercalator and combining said carrier substance with coupled intercalator with;
c) a nucleic acid to allow intercalation of said coupled intercalator with said nucleic acid.

15. The method of claim 14 wherein said coupling of carrier substance of (a) to said intercalator of (b) includes a biocleavable linkage selected from the group consisting of a hydrazone linkage, a disulfide linkage, a protected disulfide linkage, an ester linkage, an ortho ester linkage, a phosphonamide linkage, a biocleavable polypeptide, an aromatic azo linkage and an aldehyde bond.

16. The method of claim 14 wherein said nucleic acid intercalator of (b) is selected from the group consisting of photoreactive intercalators.

17. The method of claim 14 further comprising the step of coupling a targeting molecule to said carrier substance.

18. The method of claim 14 further comprising the step of coupling a transduction vector to said carrier substance.

19. The method of claim 14 further comprising the step of coupling a chloroquine substance to said carrier substance.


SEQUENCE LISTING (51 sequences)

SEQ ID NO: 1 - protein, 4 aa, Artificial Sequence
  Synthetic Construct
GFLG

SEQ ID NO: 2 - protein, 5 aa, Artificial Sequence
  Synthetic Construct
GFLFG

SEQ ID NO: 3 - protein, 4 aa, Artificial Sequence
  Synthetic Construct
GFFG

SEQ ID NO: 4 - protein, 5 aa, Artificial Sequence
  Synthetic Construct
YGGFL

SEQ ID NO: 5 - protein, 4 aa, Artificial Sequence
  Synthetic Construct
GGGF

SEQ ID NO: 6 - protein, 10 aa, Artificial Sequence
  Synthetic Construct
GFQGVQFAGF

SEQ ID NO: 7 - protein, 10 aa, Artificial Sequence
  Synthetic Construct
GFGSVQFAGF

SEQ ID NO: 8 - protein, 10 aa, Artificial Sequence
  Synthetic Construct
GFGSTFFAGF

SEQ ID NO: 9 - protein, 10 aa, Artificial Sequence
  Synthetic Construct
GLVGGAGAGF

SEQ ID NO: 10 - protein, 10 aa, Artificial Sequence
  Synthetic Construct
GGFLGLGAGF

SEQ ID NO: 11 - protein, 10 aa, Artificial Sequence
  Synthetic Construct
GFQGVQFAGF

SEQ ID NO: 12 - protein, 10 aa, Artificial Sequence
  Synthetic Construct
GFGSVQFAGF

SEQ ID NO: 13 - protein, 10 aa, Artificial Sequence
  Synthetic Construct
GLVGGAGAGF

SEQ ID NO: 14 - protein, 10 aa, Artificial Sequence
  Synthetic Construct
GGFLGLGAGF

SEQ ID NO: 15 - protein, 10 aa, Artificial Sequence
  Synthetic Construct
GFGSTFFAGF

SEQ ID NO: 16 - protein, 4 aa, Artificial Sequence
  Synthetic Construct
KALA

SEQ ID NO: 17 - DNA, 25 bp, Artificial Sequence
  Synthetic Construct
uguggaugacugaguaccugadtdt

SEQ ID NO: 18 - DNA, 25 bp, Artificial Sequence
  Synthetic Construct
ucagguacucagucauccacadtdt

SEQ ID NO: 19 - DNA, 32 bp, Artificial Sequence
  Synthetic Construct
ttttttcttttttttctcccagcgtgcgccat

SEQ ID NO: 20 - protein, 16 aa, Diptera
RQIKIWFQNRRMKWKK

SEQ ID NO: 21 - protein, 16 aa, Artificial Sequence
  Table I Transduction Peptide
KKWKMRRNQFWVKVQR

SEQ ID NO: 22 - protein, 16 aa, Artificial Sequence
  Table I Transduction Peptide
RRWRRWWRRWWRRWRR

SEQ ID NO: 23 - protein, 18 aa, Diptera
RQIKIWFQNRRMKWKKEN

SEQ ID NO: 24 - protein, 7 aa, Diptera
RRMKWKK

SEQ ID NO: 25 - protein, 13 aa, Virus
GRKKRRQRRRPPQ

SEQ ID NO: 26 - protein, 11 aa, Virus
YGRKKRRQRRR

SEQ ID NO: 27 - protein, 14 aa, Virus
PTSQSRGDPTGPKE

SEQ ID NO: 28 - protein, 17 aa, Virus
AVGAIGALFLGFLGAAG

SEQ ID NO: 29 - protein, 17 aa, Artificial Sequence
  Table I Transduction Peptide
GALFLGWLGAAGSTMGA

SEQ ID NO: 30 - protein, 30 aa, Artificial Sequence
  Table I Transduction Peptide
GALFLGFLGAAGSTMGAWSQPKSKRKVMPG

SEQ ID NO: 31 - protein, 24 aa, Artificial Sequence
  Table I Transduction Peptide
DRVIEVVQGAYRAIRNIPRRIRQG

SEQ ID NO: 32 - protein, 17 aa, Crocodile
MGLGLHLLVLAAALQGA

SEQ ID NO: 33 - protein, 27 aa, Crocodile
MGLGLHLLVLAAALQGAWSQPKKKRKV

SEQ ID NO: 34 - protein, 12 aa, Artificial Sequence
  Table I Transduction Peptide
PLSSIFSRIGDP

SEQ ID NO: 35 - protein, 27 aa, Artificial Sequence
  Table I Transduction Peptide
GWTLNSAGYLLGKINLKALAALAKKIL

SEQ ID NO: 36 - protein, 18 aa, Artificial Sequence
  Table I Transduction Peptide
RGGRLSYSRRRFSTSTGR

SEQ ID NO: 37 - protein, 16 aa, Mammalian
AAVALLPAVLLALLAP

SEQ ID NO: 38 - protein, 12 aa, Mammalian
AAVLLPVLLAAP

SEQ ID NO: 39 - protein, 15 aa, Human
VTVLALGALAGVGVG

SEQ ID NO: 40 - protein, 30 aa, Artificial Sequence
  Table I Transduction Peptide
VAYISRGGVSTYYSDTVKGRFTRQKYNKRA

SEQ ID NO: 41 - protein, 18 aa, Artificial Sequence
  Table I Transduction Peptide
KLALKLALKALKAALKLA

SEQ ID NO: 42 - protein, 30 aa, Artificial Sequence
  Table I Transduction Peptide
WEAKLAKALAKALAKHLAKALAKALKACEA

SEQ ID NO: 43 - protein, 24 aa, Mammalian
GLFEAIAGFIENGWEGMIDGGGYC

SEQ ID NO: 44 - protein, 7 aa, Artificial Sequence
  Table I Transduction Peptide
RRRRRRR

SEQ ID NO: 45 - protein, 26 aa, Artificial Sequence
  Table I Transduction Peptide
AAVALLPAVLLALLAPVQRKRQKLMP

SEQ ID NO: 46 - protein, 23 aa, Artificial Sequence
  Table I Transduction Peptide
MGLGLHLLVLAAALQGAKKKRKV

SEQ ID NO: 47 - protein, 14 aa, Artificial Sequence
  Table II Receptor Binding
TQPREEQYNSTFRV

SEQ ID NO: 48 - protein, 14 aa, Artificial Sequence
  Table II Receptor Binding
DGCSKAPKLPAALC

SEQ ID NO: 49 - protein, 8 aa, Artificial Sequence
  Table II Receptor Binding
YGGFLRRG

SEQ ID NO: 50 - protein, 3 aa, Artificial Sequence
  Table II Receptor Binding
YEE

SEQ ID NO: 51 - protein, 6 aa, Artificial Sequence
  Table II Receptor Binding
ZDFLFG